SEQUENCE LISTING (26 sequences)

SEQ ID NO: 1 - protein, 14 aa, tetanus toxin
QYIKANSKFIGITE

SEQ ID NO: 2 - protein, 21 aa, tetanus toxin
FNNFTVSFWLRVPKVSASHLE

SEQ ID NO: 3 - protein, 21 aa, Plasmodium falciparum
DIEKKIAKMEKASSVFNVVNS

SEQ ID NO: 4 - protein, 15 aa, Paramyxoviridae morbillivirus (measles virus)
LSEIKGVIVHRLEGV

SEQ ID NO: 5 - protein, 15 aa, Hepatitis B virus
FFLLTRILTIPQSLD

SEQ ID NO: 6 - protein, 20 aa, Diphtheria toxin
PVFAGANYAAWAVNVAQVID

SEQ ID NO: 7 - protein, 20 aa, Diphtheria toxin
VHHNTEEIVAQSIALSSLMV

SEQ ID NO: 8 - protein, 20 aa, Diphtheria toxin
QSIALSSLMVAQAIPLVGEL

SEQ ID NO: 9 - protein, 20 aa, Diphtheria toxin
VDIGFAAYNFVESIINLFQV

SEQ ID NO: 10 - protein, 20 aa, Diphtheria toxin
QGESGHDIKITAENTPLPIA

SEQ ID NO: 11 - protein, 20 aa, Diphtheria toxin
GVLLPTIPGKLDVNKSKTHI

SEQ ID NO: 12 - protein, 1255 aa, Homo sapiens [sequence omitted]

SEQ ID NO: 13 - protein, 1256 aa, Murinae rattus (rat)
MELAAWCRWGFLLALLPPGIAGTQVCTGTDMKLRLPASPETHLDMLRHLYQGCQVVQGNLELTYVPANASLSFLQDIQEVQGYMLIAHNQVKRVPLQRLRIVRGTQLFEDKYALAVLDNRDPQDNVAASTPGRTPEGLRELQLRSLTEILKGGVLIRGNPQLCYQDMVLWKDVFRKNNQLAPVDIDTNRSRACPPCAPACKDNHCWGESPEDCQILTGTICTSGCARCKGRLPTDCCHEQCAAGCTGPKHSDCLACLHFNHSGICELHCPALVTYNTDTFESMHNPEGRYTFGASCVTTCPYNYLSTEVGSCTLVCPPNNQEVTAEDGTQRCEKCSKPCARVCYGLGMEHLRGARAITSDNVQEFDGCKKIFGSLAFLPESFDGDPSSGIAPLRPEQLQVFETLEEITGYLYISAWPDSLRDLSVFQNLRIIRGRILHDGAYSLTLQGLGIHSLGLRSLRELGSGLALIHRNAHLCFVHTVPWDQLFRNPHQALLHSGNRPEEDCGLEGLVCNSLCAHGHCWGPGPTQCVNCSHFLRGQECVEECRVWKGLPREYVSDKRCLPCHPECQPQNSSETCFGSEADQCAACAHYKDSSSCVARCPSGVKPDLSYMPIWKYPDEEGICQPCPINCTHSCVDLDERGCPAEQRASPVTFIIATVEGVLLFLILVVVVGILIKRRRQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVREHRGRLGSQDLLNWCVQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPSSPMDSTFYRSLLEDDDMGDLVDAEEYLVPQQGFFSPDPTPGTGSTAHRRHRSSSTRSGGGELTLGLEPSEEGPPRSPLAPSEGAGSDVFDGDLAMGVTKGLQSLSPHDLSPLQRYSEDPTLPLPPETDGYVAPLACSPQPEYVNQSEVQPQPPLTPEGPLPPVRPAGATLERPKTLSPGKNGVVKDVFAFGGAVENPEYLVPREGTASPPHPSPAFSPAFDNLYYWDQNSSEQGPPPSNFEGTPTAENPEYLGLDVPV

SEQ ID NO: 14 - protein, 653 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTS

SEQ ID NO: 15 - protein, 266 aa, Homo sapiens
QNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 16 - protein, 59 aa, Homo sapiens
QNEDLGPASPLDSTFYRSLLEDDDMGDLVDAEEYLVPQQGFFCPDPAPGAGGMVHHRHR

SEQ ID NO: 17 - protein, 919 aa, Artificial Sequence
  Amino acid sequence of a fusion protein comprising the extracellular domain (ECD) and the phosphorylation domain (PD) of the human HER-2/neu protein
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 18 - protein, 712 aa, Artificial Sequence
  Amino acid sequence of a fusion protein comprising the extracellular domain (ECD) and an exemplary portion of the phosphorylation domain (?PD) of the human HER-2/neu protein
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSQNEDLGPASPLDSTFYRSLLEDDDMGDLVDAEEYLVPQQGFFCPDPAPGAGGMVHHRHR

SEQ ID NO: 19 - protein, 654 aa, Murinae rattus (rat)
MELAAWCRWGFLLALLPPGIAGTQVCTGTDMKLRLPASPETHLDMLRHLYQGCQVVQGNLELTYVPANASLSFLQDIQEVQGYMLIAHNQVKRVPLQRLRIVRGTQLFEDKYALAVLDNRDPQDNVAASTPGRTPEGLRELQLRSLTEILKGGVLIRGNPQLCYQDMVLWKDVFRKNNQLAPVDIDTNRSRACPPCAPACKDNHCWGESPEDCQILTGTICTSGCARCKGRLPTDCCHEQCAAGCTGPKHSDCLACLHFNHSGICELHCPALVTYNTDTFESMHNPEGRYTFGASCVTTCPYNYLSTEVGSCTLVCPPNNQEVTAEDGTQRCEKCSKPCARVCYGLGMEHLRGARAITSDNVQEFDGCKKIFGSLAFLPESFDGDPSSGIAPLRPEQLQVFETLEEITGYLYISAWPDSLRDLSVFQNLRIIRGRILHDGAYSLTLQGLGIHSLGLRSLRELGSGLALIHRNAHLCFVHTVPWDQLFRNPHQALLHSGNRPEEDCGLEGLVCNSLCAHGHCWGPGPTQCVNCSHFLRGQECVEECRVWKGLPREYVSDKRCLPCHPECQPQNSSETCFGSEADQCAACAHYKDSSSCVARCPSGVKPDLSYMPIWKYPDEEGICQPCPINCTHSCVDLDERGCPAEQRASPVTF

SEQ ID NO: 20 - DNA, 3768 bp, homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 21 - DNA, 3955 bp, Murinae rattus (rat)
ccgggccggagccgcaatgatcatcatggagctggcggcctggtgccgctgggggttcctcctcgccctcctgccccccggaatcgcgggcacccaagtgtgtaccggcacagacatgaagttgcggctccctgccagtcctgagacccacctggacatgctccgccacctgtaccagggctgtcaggtagtgcagggcaacttggagcttacctacgtgcctgccaatgccagcctctcattcctgcaggacatccaggaagttcagggttacatgctcatcgctcacaaccaggtgaagcgcgtcccactgcaaaggctgcgcatcgtgagagggacccagctctttgaggacaagtatgccctggctgtgctagacaaccgagatcctcaggacaatgtcgccgcctccaccccaggcagaaccccagaggggctgcgggagctgcagcttcgaagtctcacagagatcctgaagggaggagttttgatccgtgggaaccctcagctctgctaccaggacatggttttgtggaaggacgtcttccgcaagaataaccaactggctcctgtcgatatagacaccaatcgttcccgggcctgtccaccttgtgcccccgcctgcaaagacaatcactgttggggtgagagtccggaagactgtcagatcttgactggcaccatctgtaccagtggttgtgcccggtgcaagggccggctgcccactgactgctgccatgagcagtgtgccgcaggctgcacgggccccaagcattctgactgcctggcctgcctccacttcaatcatagtggtatctgtgagctgcactgcccagccctcgtcacctacaacacagacacctttgagtccatgcacaaccctgagggtcgctacacctttggtgccagctgcgtgaccacctgcccctacaactacctgtctacggaagtgggatcctgcactctggtgtgtcccccgaataaccaagaggtcacagctgaggacggaacacagcgttgtgagaaatgcagcaagccctgtgctcgagtgtgctatggtctgggcatggagcaccttcgaggggcgagggccatcaccagtgacaatgtccaggagtttgatggctgcaagaagatctttgggagcctggcatttttgccggagagctttgatggggacccctcctccggcattgctccgctgaggcctgagcagctccaagtgttcgaaaccctggaggagatcacaggttacctgtacatctcagcatggccagacagtctccgtgacctcagtgtcttccagaaccttcgaatcattcggggacggattctccacgatggcgcgtactcattgacactgcaaggcctggggatccactcgctggggctgcgctcactgcgggagctgggcagtggattggctctgattcaccgcaacgcccatctctgctttgtacacactgtaccttgggaccagctcttccggaacccacatcaggccctgctccacagtgggaaccggccggaagaggacttgtgcgtctcgagcggcttggtctgtaactcactgtgtgcccacgggcactgctgggggccagggcccacccagtgtgtcaactgcagtcatttccttcggggccaggagtgtgtggaggagtgccgagtatggaaggggctcccccgggagtatgtgagtgacaagcgctgtctgccgtgtcaccccgagtgtcagcctcaaaacagctcagagacctgctttggatcggaggctgatcagtgtgcagcctgcgcccactacaaggactcgtcctcctgtgtggctcgctgccccagtggtgtgaaaccggacctctcctacatgcccatctggaagtacccggatgaggagggcatatgccagccgtgccccatcaactgcacccactcctgtgtggatctggatgaacgaggctgcccagcagagcagagagccagcccggtgacattcatcattgcaactgtagagggcgtcctgctgttcctgatcttagtggtggtcgttggaatcctaatcaaacgaaggagacagaagatccggaagtatacgatgcgtaggctgctgcaggaaactgagttagtggagccgctgacgcccagcggagcaatgcccaaccaggctcagatgcggatcctaaaagagacggagctaaggaaggtgaaggtgcttggatcaggagcttttggcactgtctacaagggcatctggatcccagatggggagaatgtgaaaatccccgtggctatcaaggtgttgagagaaaacacatctcctaaagccaacaaagaaattctagatgaagcgtatgtgatggctggtgtgggttctccgtatgtgtcccgcctcctgggcatctgcctgacatccacagtacagctggtgacacagcttatgccctacggctgccttctggaccatgtccgagaacaccgaggtcgcctaggctcccaggacctgctcaactggtgtgttcagattgccaaggggatgagctacctggaggacgtgcggcttgtacacagggacctggctgcccggaatgtgctagtcaagagtcccaaccacgtcaagattacagatttcgggctggctcggctgctggacattgatgagacagagtaccatgcagatgggggcaaggtgcccatcaaatggatggcattggaatctattctcagacgccggttcacccatcagagtgatgtgtggagctatggagtgactgtgtgggagctgatgacttttggggccaaaccttacgatggaatcccagcccgggagatccctgatttgctggagaagggagaacgcctacctcagcctccaatctgcaccattgatgtctacatgattatggtcaaatgttggatgattgactctgaatgtcgcccgagattccgggagttggtgtcagaattttcacgtatggcgagggacccccagcgttttgtggtcatccagaacgaggacttgggcccatccagccccatggacagtaccttctaccgttcactgctggaagatgatgacatgggtgacctggtagacgctgaagagtatctggtgccccagcagggattcttctccccggaccctaccccaggcactgggagcacagcccatagaaggcaccgcagctcgtccaccaggagtggaggtggtgagctgacactgggcctggagccctcggaagaagggccccccagatctccactggctccctcggaaggggctggctccgatgtgtttgatggtgacctggcaatgggggtaaccaaagggctgcagagcctctctccacatgacctcagccctctacagcggtacagcgaggaccccacattacctctgccccccgagactgatggctatgttgctcccctggcctgcagcccccagcccgagtatgtgaaccaatcagaggttcagcctcagcctcctttaaccccagagggtcctctgcctcctgtccggcctgctggtgctactctagaaagacccaagactctctctcctgggaagaatggggttgtcaaagacgtttttgccttcgggggtgctgtggagaaccctgaatacttagtaccgagagaaggcactgcctctccgccccacccttctcctgccttcagcccagcctttgacaacctctattactgggaccagaactcatcggagcaggggcctccaccaagtaactttgaagggacccccactgcagagaaccctgagtacctaggcctggatgtacctgtatgagacgtgtgcagacgtcctgtgctttcagagtggggaaggcctgacttgtggtctccatcgccacaaagcagggagagggtcctctggccacattacatccagggcagacggctctaccaggaacctgccccgaggaacctttccttgctgcttgaa

SEQ ID NO: 22 - DNA, 20 bp, Artificial Sequence
  Immunostimulatory oligonucleotide CpG1826
tccatgacgttcctgacgtt

SEQ ID NO: 23 - DNA, 18 bp, Artificial Sequence
  Immunostimulatory oligonucleotide CpG1758
tctcccagcgtgcgccat

SEQ ID NO: 24 - DNA, 30 bp, Artificial Sequence
  Immunostimulatory CpG oligonucleotide
accgatgacgtcgccggtgacggcaccacg

SEQ ID NO: 25 - DNA, 24 bp, Artificial Sequence
  Immunostimulatory oligonucleotide CpG2006
tcgtcgttttgtcgttttgtcgtt

SEQ ID NO: 26 - DNA, 20 bp, Artificial Sequence
  Immunostimulatory oligonucleotide CpG1668
tccatgacgttcctgatgct

(19) United States
(12) Patent Application Publication
(10) Pub. No.: US 2010/0028414 A1
(43) Pub. Date: Feb. 4, 2010

(54) COMBINATION OF TYROSINE KINASE INHIBITOR AND HER-2/NEU FOR CANCER THERAPY

(76) Inventors: Claudine Elvire Marie Bruck, King of Prussia, PA (US); Catherine Marie Ghislaine Gerard, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/720,621

(22) PCT Filed: Dec. 8, 2005
(86) PCT No.: PCT/EP2005/013409

§ 371 Date: Oct. 5, 2009

(30) Foreign Application Priority Data
Dec. 10, 2004 (GB) 0427131.8

(51) Int. Cl. A61K 39/00; A61K 9/127; A61P 35/00

(52) U.S. Cl. 424/450; 424/185.1

(57) ABSTRACT

A method of treating cancer is described comprising administration of a 4-quinazolineamine and a vaccine targeting the HER-2/neu molecule, as well as a pharmaceutical combination comprising 4-quinazolineamines and a vaccine targeting the HER-2/neu molecule.

### FIELD OF THE INVENTION

The present invention relates to a combination of (a) a quinazoline derivative which is an inhibitor of the tyrosine kinases Human epidermal growth factor receptor 2 (also known as HER-2/neu or c-erbB2) and the epidermal growth factor receptor (also known as EGFR or c-erbB1) and (b) an immunogenic composition targeting the HER-2 molecule. The combination may be used in the treatment of cancer.

### BACKGROUND OF THE INVENTION

Protein tyrosine kinases catalyse the phosphorylation of specific tyrosyl residues in various proteins involved in the regulation of cell growth and differentiation. HER-2/neu and EGFR are examples of protein tyrosine kinases. Examples of inhibitors of particular protein tyrosine kinases are given in, for example, WO99/35146 (US2002 177567), incorporated herein by reference.

EGFR is a 170-kDa single-chain transmembrane glycoprotein consisting of an intracellular catalytic domain that possesses tyrosine kinase activity, an anchoring membrane spanning domain and an extracellular ligand binding region.

HER-2/neu is a member of the epidermal growth factor receptor family, a family of tyrosine kinase receptors. Examples of vaccines or immunogenic compositions targeting the HER-2/neu molecule have been described in, for example, WO00/44899 (US2002 177567) incorporated herein by reference.

HER-2/neu is a transmembrane protein with a predicted relative molecular mass of 185 kD that is about 1255 amino acids in length. HER-2/neu has an extracellular domain (ECD) of about 645 amino acids, a highly hydrophobic transmembrane domain, and a carboxy terminal intracellular domain (ICD) of about 580 amino acids. The term “PD” stands for the “phosphorylation domain” (i.e., the domain that is phosphorylated) within the intracellular domain, “ΔPD” refers to a particular fragment of the phosphorylation domain that is within the phosphorylation domain (as shown in SEQ ID NO: 16), and “KD” refers to the kinase domain that is within the intracellular domain. HER-2/neu PD is 268 amino acids in length, is intracellular, and can be phosphorylated by protein tyrosine kinases.

The HER-2/neu gene is amplified and the protein over-expressed in approximately 30% of patients with breast cancer. HER-2/neu over-expression has been described in a variety of different malignancies including breast, ovary, renal cell, prostate, pancreas, colon, non-small cell lung, gastric, salivary gland, bladder and oral squamous cell. In patients with breast cancer HER-2/neu over-expression is a poor prognostic factor and appears to be predictive for resistance to some chemotherapeutic agents.

HER-2/neu vaccines based on HER-2/neu DNA and Her2 peptides have been shown to induce T cell immunity to HER-2/neu in animal models and in human vaccine trials. Furthermore, Trastuzumab (Herceptin®, a humanized monoclonal antibody to HER-2/neu was found to be effective for treatment of metastatic breast cancer.

### Statement of Invention

In a first aspect of the present invention there is provided a method of treating cancer in a mammal, comprising: administering to said mammal a therapeutically effective amount of
(a) a compound of formula I, II, III or IV as described herein, and/or salts, solvates or physiologically functional derivatives thereof, in which R₁ is Cl or Br; X is CH, N, or CF; and Het is thiazole or furan; and
(b) an immunogenic composition comprising isolated protein comprising at least one epitope from the HER-2/neu protein, or a polynucleotide encoding such a protein.

In a second aspect of the present invention there is provided a pharmaceutical combination, comprising therapeutically effective amounts of:
(a) a compound of formula I, II, III or IV as described herein, and/or salts, solvates or physiologically functional derivatives thereof, in which R₁ is Cl or Br; X is CH, N, or CF; and Het is thiazole or furan; and
(b) an immunogenic composition comprising isolated protein comprising at least one epitope from the HER-2/neu protein, or a polynucleotide encoding such a protein.

In a third aspect of the present invention there is provided a use of a pharmaceutical combination as described herein in the preparation of a medicament for treatment of cancer.

In a fourth aspect of the present invention there is provided a use of a pharmaceutical combination comprising therapeutically effective amounts of a compound of formula I, II, III or IV as described herein and/or salts, solvates or physiologically functional derivatives thereof in the preparation of a medicament for treatment of cancer in an individual, wherein the individual has been administered with an immunogenic composition comprising at least one epitope from the HER-2/neu protein, or a polynucleotide encoding such a protein, as described herein.

Surprisingly, the inventors have found that the combination of component (a) of the present invention, together with administration of sera obtained following immunisation with an immunogenic composition (b) of the present invention, resulted in growth inhibition of breast cancer cells. This synergistic effect of the two components was much more pronounced than inhibition shown with single agent treatment.

### BRIEF DESCRIPTION OF THE FIGURES

FIG. 1 shows the full length amino acid sequence of the human HER-2/neu protein (SEQ ID NO:12).

FIG. 2 shows the full length amino acid sequence of the rat HER-2/neu protein (SEQ ID NO:13). The kinase domain spans the region from amino acid 721 to amino acid 998, inclusively.

FIG. 3 shows the amino acid sequence of the extracellular HER-2/neu protein (SEQ ID NO:14).

FIG. 4 shows the amino acid sequence of the phosphorylation domain (PD) of the human HER-2/neu protein (SEQ ID NO:15).

FIG. 5 shows an example of the amino acid sequence of a portion of the phosphorylation domain (ΔPD) of the human HER-2/neu protein (SEQ ID NO:16).

FIG. 6 shows the amino acid sequence of a fusion protein comprising the extracellular domain (ECD) and the phosphorylation domain (PD) of the human HER-2/neu protein (SEQ ID NO:17).

FIG. 7 shows the amino acid sequence of a fusion protein comprising the extracellular domain (ECD) and an exemplary portion of the phosphorylation domain (ΔPD) of the human HER-2/neu protein (SEQ ID NO:18).

FIG. 8 shows the amino acid sequence of the extracellular domain (ECD) of the rat HER-2/neu protein (SEQ ID NO:19).

FIG. 9 shows the full length nucleotide sequence (SEQ ID NO:20) of a DNA molecule encoding the human HER-2/neu protein. The full length nucleotide sequence is described in WO 96/30514 (U.S. Pat. No. 5,726,023) and the fusion proteins are described in WO00/44899 (US2002/0177567), the disclosures of which are incorporated by reference herein in their entirety.

FIG. 10 shows the full length nucleotide sequence (SEQ ID NO:21) of a DNA molecule encoding the rat HER-2/neu protein. This full length nucleotide sequence is described by Bargmann et al. (1986) Nature, 319:226-30, and GENBANK/X03362, the disclosures of which are incorporated by reference herein in their entirety.

FIG. 11A shows Anti-proliferative effect of anti HER2/neu polyclonal antibodies in combination with Lapatinib on the proliferation of BT474 human breast cancer cells.

FIG. 11B shows Anti-proliferative effect of anti HER2/neu polyclonal antibodies in combination with Lapatinib on the proliferation of SKBR3 human breast cancer cells.

FIG. 12A (top panel) shows the results of treating exponentially growing BT474 cells with (i) DMSO alone (negative control for lapatinib) (ii) lapatinib (0.1 μM), (iii) pAb (100 μg/ml), (iv) lapatinib (0.1 μM) and pAb (100 μg/ml), (v) TA2021 (100 μg/ml), (vi) lapatinib (0.1 μM) and TA2021 (100 μg/ml), (vii) trastuzumab (10 μg/ml), or (viii) lapatinib (0.1 μM) and trastuzumab (10 μg/ml). After 72 hr, apoptosis was assessed using annexin V staining and flow cytometry.

FIG. 12E shows similar results to FIG. 12A.

Steady state protein levels of activated phospho-ErbB2 (p-ErbB2), total ErbB2, and survivin were also assessed after 72 hr using Western blot (bottom panel, where lane 1 is DMSO alone; lane 2 is lapatinib (0.1 μM); lane 3 is pAb (100 μg/ml); lane 4 is lapatinib (0.1 μM) and pAb (100 μg/ml); lane 5 is TA2021 (100 μg/ml); lane 6 is lapatinib (0.1 μM) and TA2021(100 μg/ml); lane 7 is trastuzumab (10 μg/ml); and lane 8 is lapatinib (0.1 μM) and trastuzumab (10 μg/ml). Actin steady state protein levels served as a control for equal loading of protein. BT474 cells treated with vehicle (DMSO) or TA2021 served as controls for lapatinib and pAb, respectively.

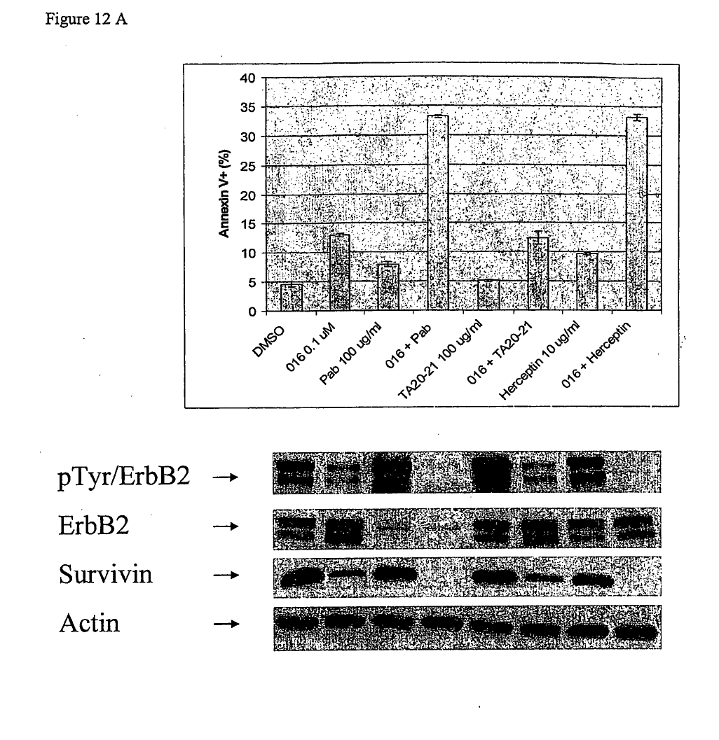

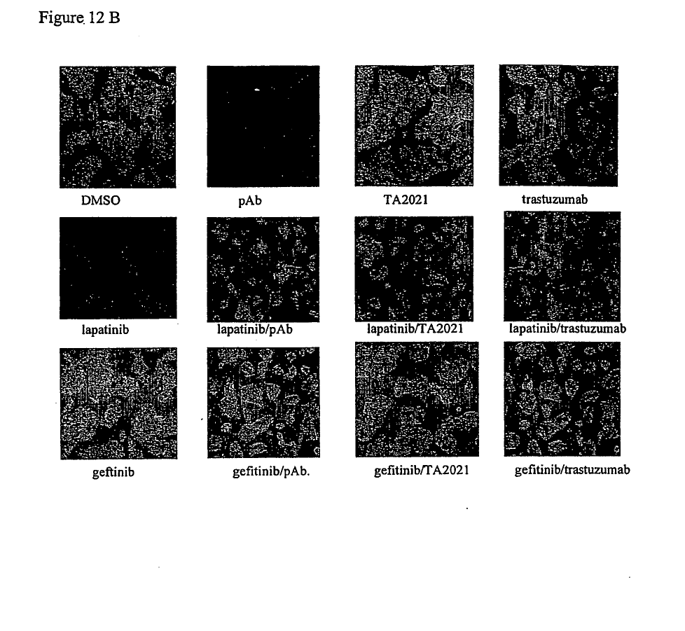

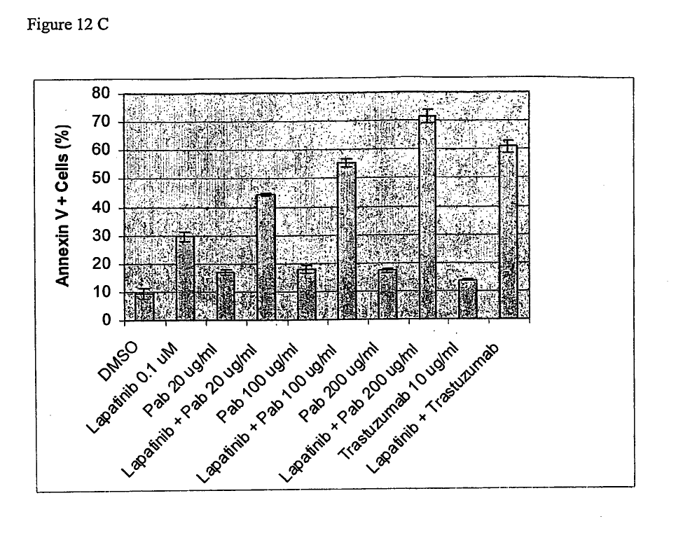

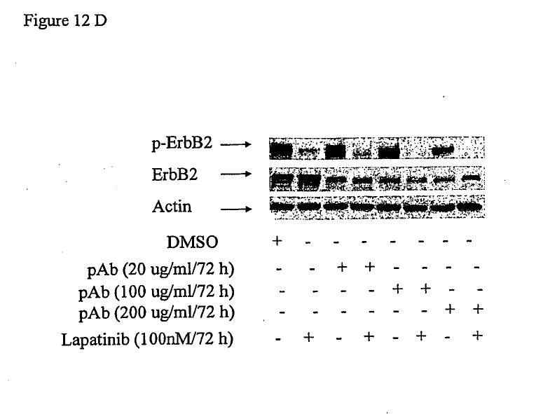

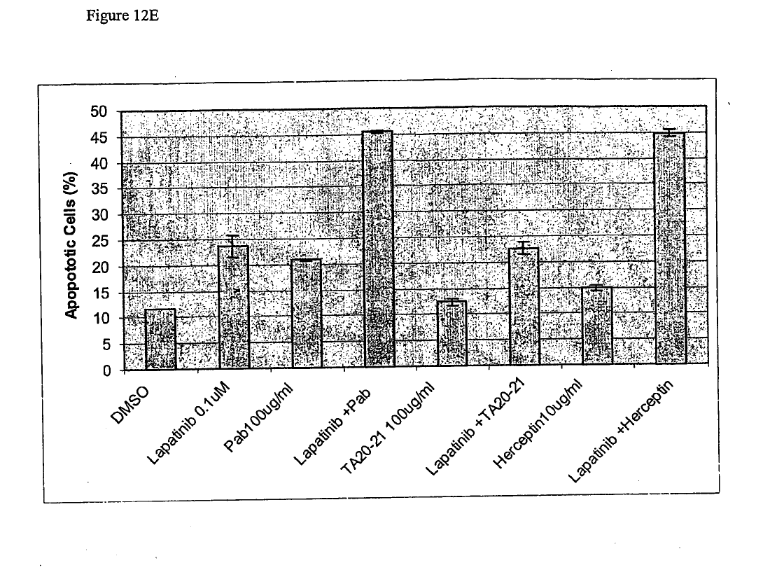

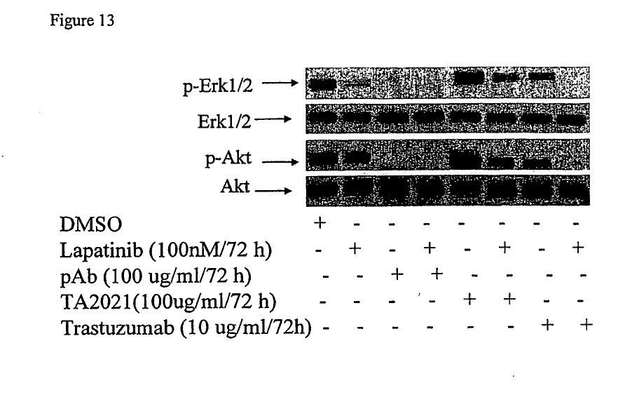

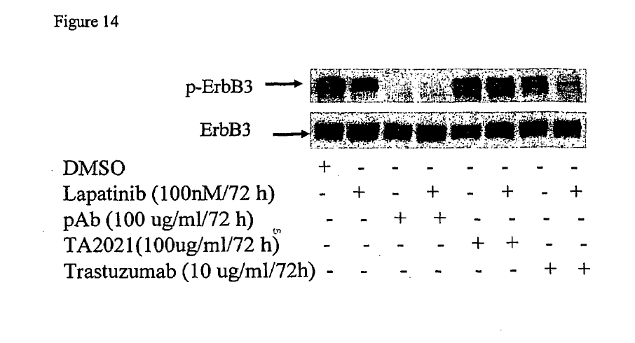

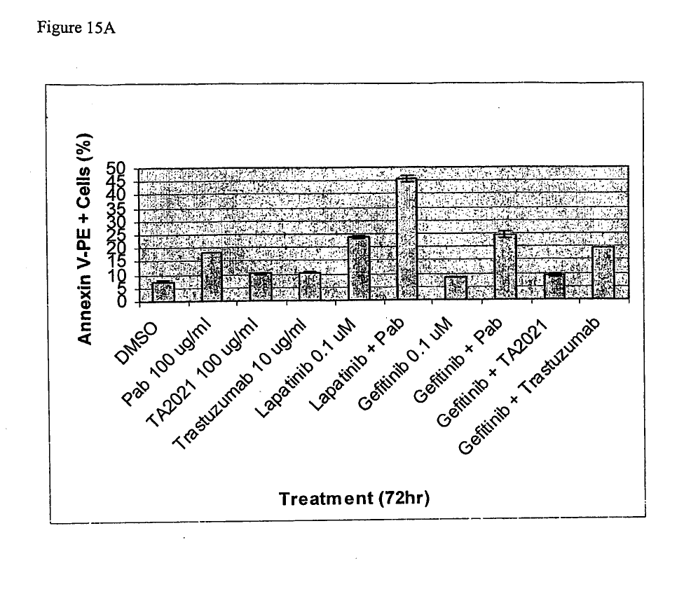

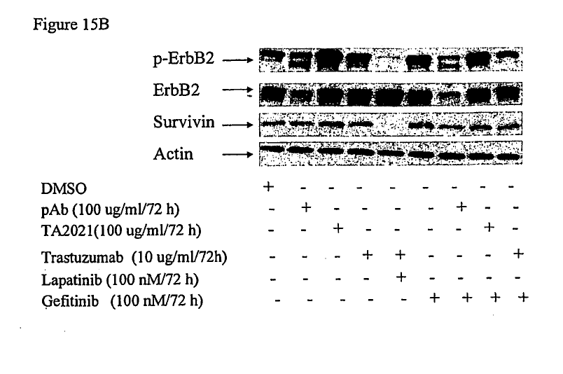

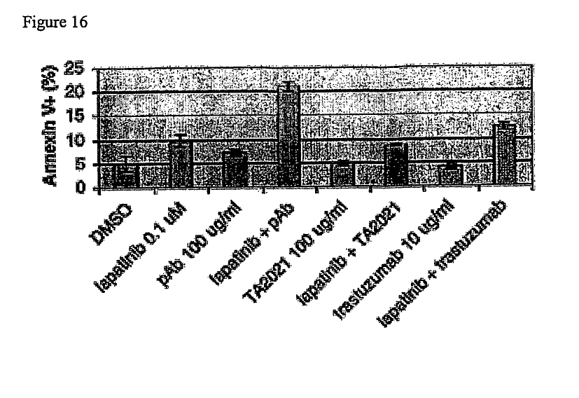

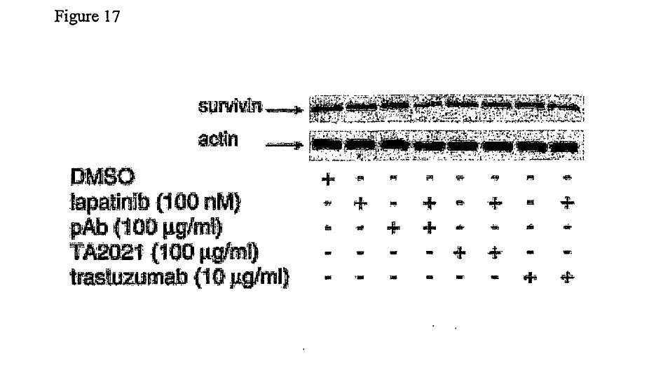

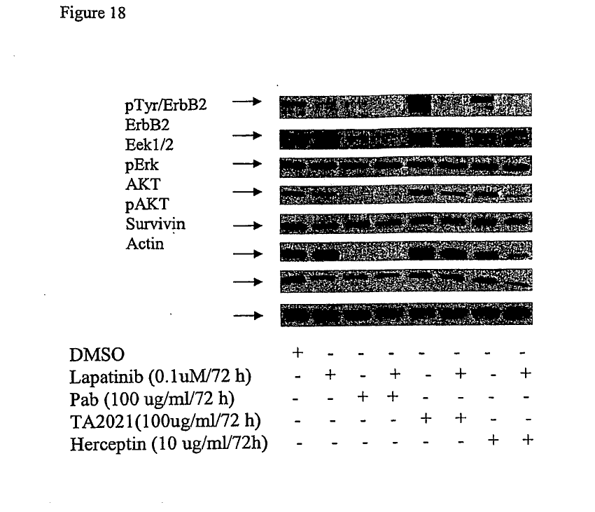

FIG. 12B shows the effects of specified treatment conditions on BT474 cell growth (after 72 hr) using contrast phase microscopy. Treatment conditions included those as described for FIG. 12A, and additionally, gefitinib (Iressa) (0.1 μM); gefitinib (0.1 μM) and pAB (100 μg/ml); gefitinib (0.1 μM) and TA2021 (100 μg/ml); and gefitinib 0.1 μM and trastuzumab (10 μg/ml).

FIG. 12C shows the effects of increasing concentrations of pAb either alone or in combination with lapatinib (100 nM) on apoptosis in BT474 cells using annexin V staining and flow cytometry.

FIG. 12D shows steady-state protein levels of total ErbB2 and p-ErbB2 in response to increasing amounts of pAb, either alone or in combination with lapatinib (100 nM) as assessed by Western blot. Actin steady state protein levels served as a control for equal loading of protein.

FIG. 12E shows similar results to FIG. 12A

FIG. 13 shows the activation-state of Erk1/2 and Akt is modulated in response to lapatinib and vaccine-induced anti-HER-2/neu antibodies (pAb). Exponentially growing BT474 cells were cultured for 72 hr under the treatment conditions described. Steady state protein levels of total Erk1/2, activated phospho-Erk1/2, total Akt, and activated phospho-Akt were assessed by Western blot. Cells treated with either vehicle (DMSO) or TA2021 alone served as controls.

FIG. 14 shows the effects of lapatinib and vaccine-induced anti-HER-2/neu antibodies (pAb) on the activation state of ErbB3. BT474 cells were cultured under various treatment conditions, as shown. After 72 hr, cell lysates were collected and steady state protein levels of total ErbB3 and activated phospho-ErbB3 assessed by Western blot.

FIG. 15A shows the effects of the indicated treatment conditions on apoptosis in BT474 cells. Apoptosis was assessed using annexin V staining and flow cytometry.

FIG. 15B shows Western blot analysis of steady state protein levels of total ErbB2, p-ErbB2, and survivin after 72 hr in BT474 cells cultured under the indicated treatment conditions.

FIG. 15C shows the effects of the indicated treatment conditions on steady state protein levels of total Erk1/2, p-Erk1/2, total Akt, and p-Akt assessed by Western blot after 72 hr of treatment.

FIG. 16 shows the apoptotic effects of treating SKBR3 cells with (i) DMSO alone (negative control for lapatinib) (ii) lapatinib (0.1 μM), (iii) pAb (100 μg/ml), (iv) lapatinib (0.1 μM) and pAb (100 μg/ml); (v) TA2021 (100 μg/ml), (vi) lapatinib (0.1 μM) and TA2021 (100 μg/ml), (vii) trastuzumab (10 μg/ml), or (viii) lapatinib (0.1 μM) and trastuzumab (10 μg/ml). After 72 hr, apoptosis was assessed using annexin V staining and flow cytometry.

FIG. 17 shows the effects on survivin protein, when SKBR3 cells were treated for 72 hours with (i) DMSO alone (negative control for lapatinib) (ii) lapatinib (0.1 μM), (iii) pAb (100 μg/ml), (iv) lapatinib (0.1 μM) and pAb (100 μg/ml); (v) TA2021 (100 μg/ml), (vi) lapatinib (0.1 μM) and TA2021 (100 μg/ml), (vii) trastuzumab (10 μg/ml), or (viii) lapatinib (0.1 μM) and trastuzumab (10 μg/ml).

FIG. 18 shows the effects of lapatinib and anti-Her-2/neu antibodies on pTyr/ErbB12 and down-stream biomarkers in SkbR3 cells, as indicated.

### DETAILED DESCRIPTION OF THE INVENTION

Throughout this specification, unless the context requires otherwise, the words “comprise” and “include” or variations such as “comprising”, “comprises”, “including”, “includes”, etc., are to be construed inclusively, that is, use of these words will imply the possible inclusion of integers or elements not specifically recited.

It should be noted that all references and publications referred to throughout the specification are incorporated herein by reference.

Additionally, the term “protein” is used herein interchangeably with “polypeptide” or “peptide”.

### Component (a)

The following structure represents a compound of formula I:

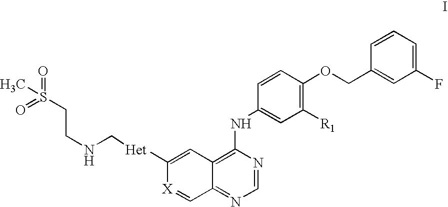

wherein R₁ is Cl or Br; X is CH, N, or CF; and Het is thiazole or furan.

In one embodiment, R₁ is Cl; X is CH; and Het is furan, and component (a) may be a compound of Formula II and/or salts, solvates or physiologically functional derivatives thereof.

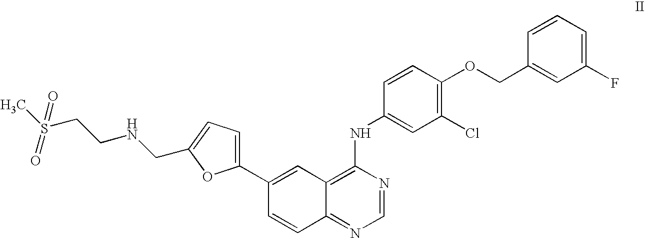

The compound of formula II has the chemical name N-{3-Chloro-4-[(3-fluorobenzyl) oxy]phenyl}-6-[5-({[2-(methanesulphonyl)ethyl]amino}methyl)-2-furyl]-4-quinazolmamine.

In another embodiment, R₁ is Cl; X is CH; and Het is thiazole, and component (a) may be a compound of formula III and/or salts, solvates or physiologically functional derivatives thereof.

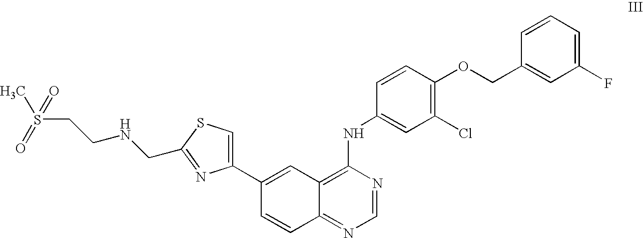

The compound of formula III is (4-(3-Fluoro-benzyloxy)-3-chlorophenyl)-(6-(2-((2-methanesulphonyl-ethylamino)-methyl)-thiazol-4-yl)quinazolin-4-yl)-amine.

In a further embodiment, R₁ is Br; X is CH; and Het is furan, and component (a) may be a compound of formula IV and/or salts, solvates or physiologically functional derivatives thereof.

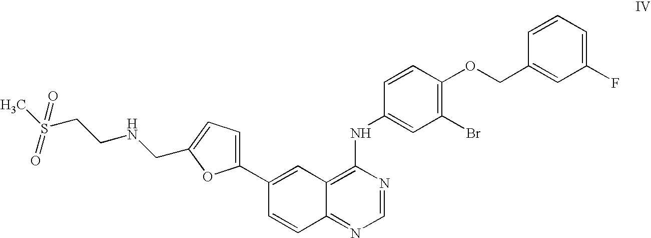

The compound of formula IV is (4-(3-Fluoro-benzyloxy)-3-bromophenyl)-(6-(5-((2-methanesulphonyl-ethylamino)-methyl)-furan-2-yl)quinazolin-4-yl)-amine.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Suitably, component (a) is administered in an effective amount and/or a therapeutically effective amount.

As used herein, the term “physiologically functional derivative” refers to any pharmaceutically acceptable derivative of a compound of Formulae I, II, III, or IV, for example, an ester or an amide, which upon administration to a mammal is capable of providing (directly or indirectly) a compound of Formulae I, II, III, or IV or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula I, II, III, or IV or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. The solvent used may be a pharmaceutically acceptable solvent. Examples of suitable pharmaceutically acceptable solvents include water, ethanol and acetic acid. In one embodiment the solvent used is water.

Compounds (a) of formulae I, II, III and IV have the ability to crystallize in more than one form, a characteristic, which is known as polymorphism, and it is understood that such polymorphic forms (“polymorphs”) are within the scope of formulae I, II, III and IV. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

Typically, the salts of the compounds of formula I, II, III, or IV are pharmaceutically acceptable salts. Salts encompassed within the term “pharmaceutically acceptable salts” refer to non-toxic salts of the compounds of this invention. Salts of the compounds of formula I, II, III, or IV may comprise acid addition salts derived from a nitrogen on a substituent in the compound of formula I, II, III, or IV. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate and ditosylate, triethiodide, trimethylarnmonium and valerate. Other salts, which are not pharmaceutically acceptable, may be useful in the preparation of compounds of this invention and these form a further aspect of the invention. Furthermore, such salt may be in anhydrous or hydrated form. In one embodiment, the compound of formula I, II, III, or IV is a hydrochloride or ditosylate salt, for example a ditosylate salt, for example the monohydrate of the ditosylate salt.

The side chain CH₃SO₂CH₂CH₂NHCH₂ of the compounds of formula I, II, III, or IV may be linked to any suitable position of the group Het. Similarly, the phenyl group of the quinazoline core may be linked to any suitable position of the group Het.

In one embodiment of the present invention, component (a) may be Lapatinib ditosylate (GSK572016; Lapatinib) (GSK), either anhydrous or hydrated form, such as the monohydrate of the ditosylate salt.

### Component (b)

As used herein, the term “immunogenic composition” encompasses any composition which, when administered to a suitable mammalian subject, has the ability to induce an immune response in that subject to at least one portion of HER-2/Neu As used herein, the term “immunogen” refers to the component of the composition which has the ability to induce an immune response to at least one portion of HER-2/Neu.

Component (b) may comprise an isolated protein comprising at least one epitope from the HER-2/neu protein, or a polynucleotide encoding such a protein. In one embodiment of the present invention, the at least one epitope is 9 or more amino acids in length.

The HER-2/neu molecule described herein may be rat, mouse, non-human primate, human or a hybrid thereof. In one embodiment, the HER-2/neu molecule described herein may be the entire protein, or a hybrid thereof. In one embodiment of the present invention, the HER-2/neu is human.

In one embodiment of the present invention, component (b) as described herein may comprise an isolated protein comprising at least one epitope from the HER-2/neu extracellular domain, or a polynucleotide encoding such a protein. In one embodiment of the present invention, the at least one epitope is 9 or more amino acids in length.

In one embodiment of the present invention, component (b) comprises an isolated protein comprising or consisting of the HER-2/neu extracellular domain, or a polynucleotide encoding such a protein.

In one embodiment of the present invention, component (b) as described herein may comprise an isolated protein comprising at least one epitope from the HER-2/neu intracellular domain, or a polynucleotide encoding such a protein. In one embodiment of the present invention, the at least one epitope is 9 or more amino acids in length.

In an alternative embodiment, component (b) comprises an isolated protein comprising or consisting of the HER-2/neu intracellular domain, or a polynucleotide encoding such a protein.

In an alternative embodiment of the present invention, component (b) comprises a fusion protein comprising at least one epitope from the HER-2/neu protein, for example from the extracellular domain, or a polynucleotide encoding such a protein. Examples of fusion proteins which may be used in the present invention are described in WO00/44899 (US2002 177567), incorporated herein by reference.

The term “fusion protein” refers to a protein comprising at least two peptides or polypeptides. In one embodiment of a fusion protein, one peptide or polypeptide is from one protein sequence or domain and the other peptide or polypeptide is from another protein sequence or domain. The peptides or polypeptides may be covalently linked, for example via a covalent linker, e.g., an amino acid linker, such as a polyglycine linker, or another type of chemical linker, e.g., a carbohydrate linker, a lipid linker, a fatty acid linker, a polyether linker, e.g., PEG, etc. (See, e.g., Hermanson, Bioconjugate techniques (1996)).

In a further embodiment of the present invention, component (b) may comprise a Her2/neu protein and/or epitope thereof as described herein, conjugated to a carrier molecule (for example using chemical conjugation techniques) or fused to a carrier molecule (for example to form a recombinant fusion protein comprising Her2/neu protein and/or epitope thereof and the carrier). The carrier may provide T-cell help for generation of an immune response to the HER-2/neu molecule.

A non-exhaustive list of carriers which may be used in the present invention includes: Keyhole Limpet Haemocyanin (KLH), serum albumins such as bovine or human serum albumin (BSA or HSA), ovalbumin (OVA), inactivated bacterial toxins such as tetanus toxoid (TT) or diphtheria toxoid (DT), or recombinant fragments thereof (for example, Domain 1 of Fragment C of TT, or the translocation domain of DT), the purified protein derivative of tuberculin (PPD). In an embodiment of the invention in which the carrier protein is of animal-origin, such as KLH or a serum albumin, the carrier protein may be recombinantly derived.

In one embodiment of the invention the carrier may be Protein D from Haemophilus influenzae (EP0594610B1 incorporated herein by reference). Protein D is an IgD-binding protein from Haemophilus influenzae and has been patented by Forsgren (WO 91/18926, granted EP 0 594 610 B1 incorporated herein by reference). In some circumstances, for example in recombinant immunogen expression systems it may be desirable to use fragments of protein D, for example Protein D ⅓rd (comprising the N-terminal 100-110 amino acids of protein D (GB 9717953.5 (U.S. Pat. No. 6,342,224) incorporated herein by reference)).

In one embodiment of the present invention the carrier may be a “T-cell helper (Th) epitope” or “T-helper epitope”, which is a peptide able to bind to an MHC molecule and stimulate T-cells in an animal species. The T-helper epitope may be a foreign or non-self epitope. T-cell epitopes may be promiscuous epitopes, ie. epitopes that bind to a substantial fraction of MHC class II molecules in an animal species or population (Panina-Bordignon et al, EJI. 1989, 19:2237-2242; Reece et al, JI 1993, 151:6175-6184 incorporated herein by reference).

Th-epitopes that are promiscuous are highly and broadly reactive in animal and human populations with widely divergent MHC types (Partidos et al. (1991) “Immune Responses in Mice Following Immunisation with chimaeric Synthetic Peptides Representing B and T Cell Epitopes of Measles Virus Proteins” J. of Gen. Virol. 72:1293-1299; U.S. Pat. No. 5,759,551, incorporated herein by reference.). The Th domains that may be used in accordance with the present invention have from about 10 to about 50 amino acids, for example from about 10 to about 30 amino acids. When multiple Th epitopes are present, these may all be the same (ie the epitopes are homologous) or a combination of more than one type of epitope may be used (ie the epitopes are heterogeneous).

Th epitopes include as examples, pathogen derived epitopes such as Hepatitis surface or core (peptide 50-69, Ferrari et al., J. Clin. Invest, 1991, 88, 214-222) antigen Th epitopes, Pertussis toxin Th epitopes, tetanus toxin Th epitopes (such as P2 (EP 0 378 881 B1 incorporated herein by reference) and P30 (WO 96/34888, WO 95/31480, WO 95/26365 incorporated herein by reference), measles virus F protein Th epitopes, Chlamydia trachomatis major outer membrane protein Th epitopes (such as P11, Stagg et al., Immunology, 1993, 79, 1-9), Yersinia invasin, diphtheria toxoid, influenza virus haemagluttinin (HA), and P. falciparum CS antigen.

Other Th epitopes are described in the literature, including: WO 98/23635; Southwood et al., 1998, J. Immunol., 160: 3363-3373; Sinigaglia et al., 1988, Nature, 336: 778-780; Rammensee et al., 1995, Immunogenetics, 41: 4, 178-228; Chicz et al., 1993, J. Exp. Med., 178:27-47; Hammer et al., 1993, Cell 74:197-203; and Falk et al., 1994, Immunogenetics, 39: 230-242, U.S. Pat. No. 5,759,551; Cease et al., 1987, PNAS 84, 4249-4253; Partidos et al., J. Gen. Virol, 1991, 72, 1293-1299; WO 95/26365 and EP 0 752 886 B. The T-cell epitope can also be an artificial sequence such as a Pan D-R peptide “PADRE” (WO95/07707 (U.S. Pat. No. 6,675,428) incorporated herein by reference). In one embodiment of the present invention, the carrier used is PADRE.

The T-cell epitope may be selected from the group of epitopes that will bind to a number of individuals expressing more than one MHC II molecules in humans. For example, epitopes that are specifically contemplated are P2 and P30 epitopes from TT (Panina-Bordignon Eur. J. Immunol 1989 19 (12) 2237). In one embodiment the heterologous T-cell epitope is P2 or P30 from TT.

The P2 epitope has the sequence QYIKANSKFIGITE (SEQ ID No: 1) and corresponds to amino acids 830-843 of the Tetanus toxin.

The P30 epitope (residues 947-967 of Tetanus Toxin) has the sequence FNNFTVSFWLRVPKVSASHLE (SEQ ID No: 2); the FNNFTV sequence may optionally be deleted.

Other universal T epitopes are derivable from the circumsporozoite protein from Plasmodium falciparum—in particular the region 378-398 having the sequence DEKKIAKMEKASSVFNVVNS (SEQ ID No: 3) (Alexander J, (1994) Immunity 1 (9), p 751-761).

Another epitope which may be used is derived from Measles virus fusion protein at residue 288-302 having the sequence LSEIKGVIVHRLEGV (SEQ ID No: 4) (Partidos C D, 1990, J. Gen. Virol 71(9) 2099-2105).

Yet another epitope which may be used is derived from hepatitis B virus surface antigen, in particular amino acids, having the sequence FFLLTRILTIPQSLD (SEQ ID No: 5).

Another set of epitopes which may be used is derived from diphtheria toxin. Four of these peptides (amino acids 271-290, 321-340, 331-350, 351-370) map within the T domain of fragment B of the toxin, and the remaining 2 map in the R domain (411-430, 431-450):

| | |
|---|---|
| PVFAGANYAAWAVNVAQVID | (SEQ ID No: 6) |
| VHHNTEEIVAQSIALSSLMV | (SEQ ID No: 7) |
| QSIALSSLMVAQAIPLVGEL | (SEQ ID No: 8) |
| VDIGFAAYNFVESIINLFQV | (SEQ ID No: 9) |
| QGESGHDIKITAENTPLPIA | (SEQ ID No: 10) |
| GVLLPTIPGKLDVNKSKTHI | (SEQ ID No: 11) |

(Raju R., Navaneetham D., Okita D., Diethelm-Okita B., McCormick D., Conti-Fine B. M. (1995) Eur. J. Inununol. 25: 3207-14.)

The immunogenic composition of the present invention may, therefore, comprise a Her2/neu protein and/or epitope thereof as described herein, and carriers and/or Th epitopes as described herein either as chemical conjugates or as purely synthetic peptide constructs. The immunogen may be joined to the Th epitopes via a spacer (e.g., Gly-Gly) at either the N- or C-terminus of the immunogen.

One or more carrier(s) and/or promiscuous Th epitope(s) may be included. In one embodiment the immunogenic composition may comprise between 2 to 5 carriers and/or Th epitopes.

In one embodiment, the immunogen may be directly conjugated to liposome carriers, which may additionally comprise immunogens capable of providing T-cell help.

### Conjugation or Fusion Protein

The carrier or Th epitope may be coupled using a method of conjugation well known in the art. Thus, for example, for direct covalent coupling it is possible to utilise a carbodiimide, glutaraldehyde or (N-[γ-maleimidobutyryloxy]succinimide ester, utilising common commercially available heterobifunctional linkers such as CDAP and SPDP (using manufacturers instructions). After the coupling reaction, the conjugate may easily be isolated and purified by means of a dialysis method, a gel filtration method, a fractionation method etc. Conjugates formed by use of gluteraldehyde or maleimide chemistry may be used in the present invention. In one embodiment, maleimide chemistry may be used.

Alternatively, the carrier or Th epitope may be fused to the Her-2/neu protein or epitope as described herein. For example, EP0421635B (incorporated herein by reference) describes the use of chimaeric hepadnavirus core antigen particles to present foreign peptide sequences in a virus-like particle. As such, fusion molecules may comprise immunogen of the present invention presented in chimaeric particles consisting of e.g. hepatitis B core antigen. Alternatively, the recombinant fusion proteins may comprise immunogen and NS1 of the influenza virus.

For any recombinantly expressed protein which forms part of component (b) of the present invention, the nucleic acid which encodes said protein also forms an aspect of the present invention.

In one embodiment of the present invention, the fusion protein may be the expression of genetically engineered fusion partners, optionally via a linker sequence.

Polypeptides forming the fusion protein may be linked C-terminus to N-terminus. Alternatively, they may be linked C-terminus to C-terminus, N-terminus to N-terminus, or N-terminus to C-terminus. The polypeptides of the fusion protein may be in any order. The terms “polypeptide” and “fusion protein” may also refer to conservatively modified variants, polymorphic variants, alleles, mutant, sub-sequences and interspecies homologues of the polypeptides, or the polypeptides that make up a fusion protein.

Fusion proteins may be produced by covalently linking a chain of amino acids from one protein sequence to a chain of amino acids from another protein sequence, e.g., by preparing a recombinant polynucleotide contiguously encoding the fusion protein. Fusion proteins can comprise 2, 3, 4 or more different chains of amino acids from the same or different species. The different chains of amino acids in a fusion protein may be directly spliced together or may be indirectly spliced together via a chemical linking group or an amino acid linking group. The fusion protein may optionally comprise other components, as described in more detail herein.

The conjugate or fusion protein may be substantially biologically inactive.

In one embodiment, the HER-2/neu protein or epitope described herein may be linked or fused to recombinant GM-CSF, for example human GM-CSF. In a further embodiment, a composition comprising a HER-2/neu protein or epitope as described herein may further comprise GM-CSF.

As used herein, the term “HER-2/neu ECD-ICD fusion protein,” also referred to herein as “ECD-ICD” or “ECD-ICD fusion protein,” refers to a fusion protein or fragment thereof which comprises (i) an amino acid sequence comprising or consisting of the extracellular domain or fragments thereof; and (ii) an amino acid sequence comprising or consisting of the intracellular domain or fragments thereof of the HER-2/neu protein.

As used herein, the term “HER-2/neu ECD-PD fusion protein”, also referred to as “ECD-PD” or “ECD-PD fusion protein”, refers to a fusion protein or fragment thereof which comprises (i) an amino acid sequence comprising or consisting of the extracellular domain or fragments thereof; and (ii) an amino acid sequence comprising or consisting of the phosphorylation domain or fragments thereof of the HER-2/neu protein.

As used herein, the term “HER-2/neu ECD-ΔPD fusion protein,” also referred to as “ECD-ΔPD” or “ECD-ΔPD fusion protein,” refers to a fusion protein or fragment thereof which comprises (i) an amino acid sequence comprising or consisting of the extracellular domain or fragments thereof; and (ii) an amino acid sequence comprising or consisting of the ΔPD domain or fragments thereof of the HER-2/neu protein.

In one embodiment, component (b) comprises an immunogenic composition comprising or consisting of the fusion protein “ECD-ICD”, or a polynucleotide encoding the fusion protein.

In an alternative embodiment, component (b) comprises an immunogenic composition comprising or consisting of the fusion protein “ECD-PD”, or a polynucleotide encoding the fusion protein.

In a further alternative embodiment, component (b) comprises an immunogenic composition comprising or consisting of the fusion protein “ECD-ΔPD”, or a polynucleotide encoding the fusion protein.

In one embodiment of the present invention, the fusion protein does not include a substantial portion of the HER-2/neu transmembrane domain. In a further embodiment, the fusion protein does not include any of the HER-2/neu transmembrane domain.

ECD-ICD fusion proteins and the ECD-PD fusion proteins of the invention may be soluble, secreted and stable in culture media.

HER-2/neu proteins as described herein are understood to include fragments thereof, homologs thereof and functional equivalents thereof (collectively referred to as “variants”), such as those in which one or more amino acids is inserted, deleted or replaced by other amino acid(s) or non-amino acid(s) which, in some embodiments of the invention, either (i) increase the elicitation or enhancement of an immune response as compared to the HER-2/neu protein, or (ii) do not substantially affect elicitation or enhancement of an immune response as compared to the HER-2/neu protein (e.g., variant stimulates a response by helper T cells or cytotoxic T cells or stimulates the production of antibodies).

Examples of variants including exemplary fragments, homologs and functional equivalents of the HER-2/neu ECD-ICD fusion protein and HER-2/neu ECD-PD fusion protein are described in more detail in WO00/44899 (US2002/0177567), incorporated herein by reference. Variants can be “substantially identical” or “substantially similar” to a fusion protein comprising native polypeptide components, and retain the ability to stimulate an immune response. Examples of variants which may be used and methods of determining such variants are described in WO00/44899 (US2002/0177567), incorporated herein by reference.

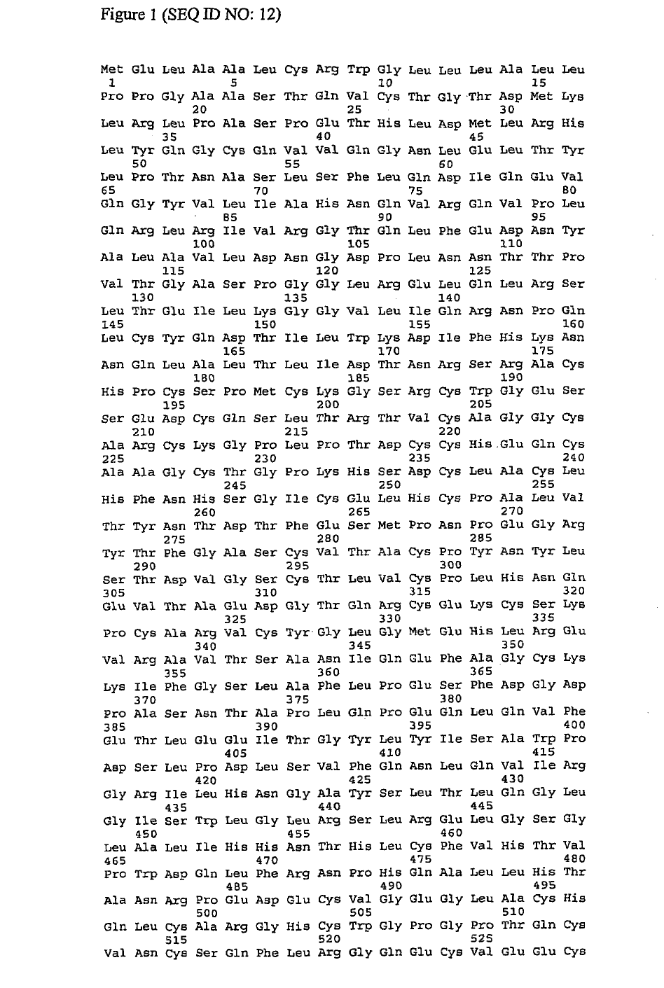

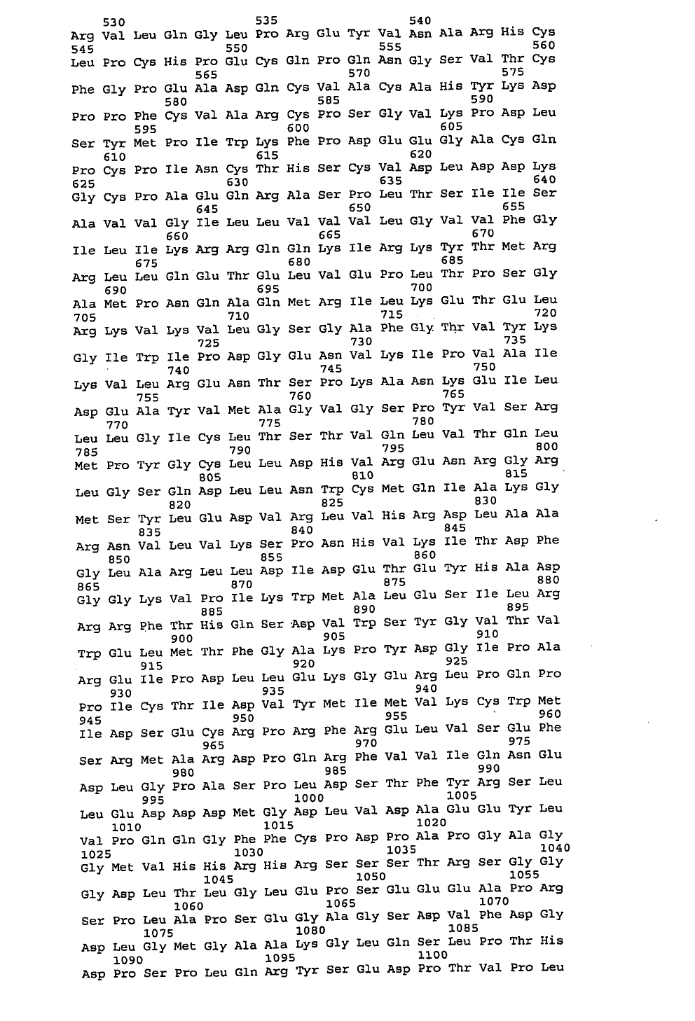

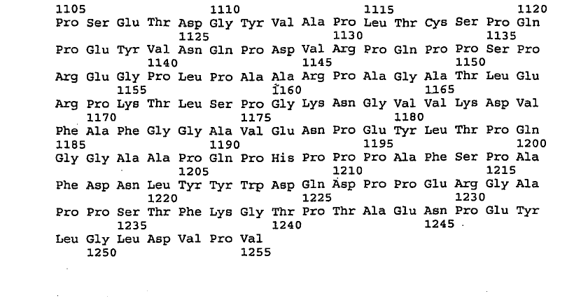

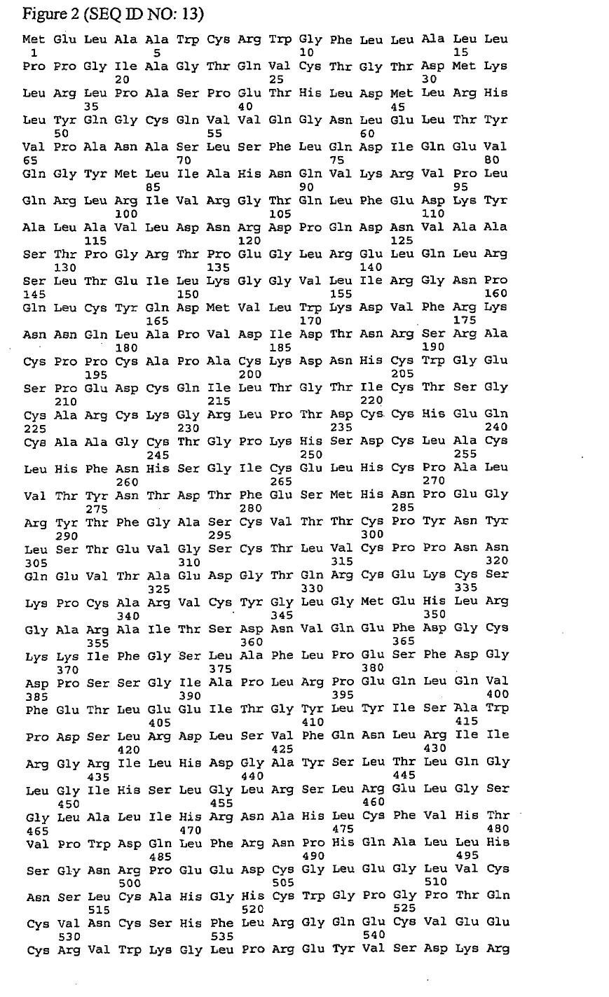

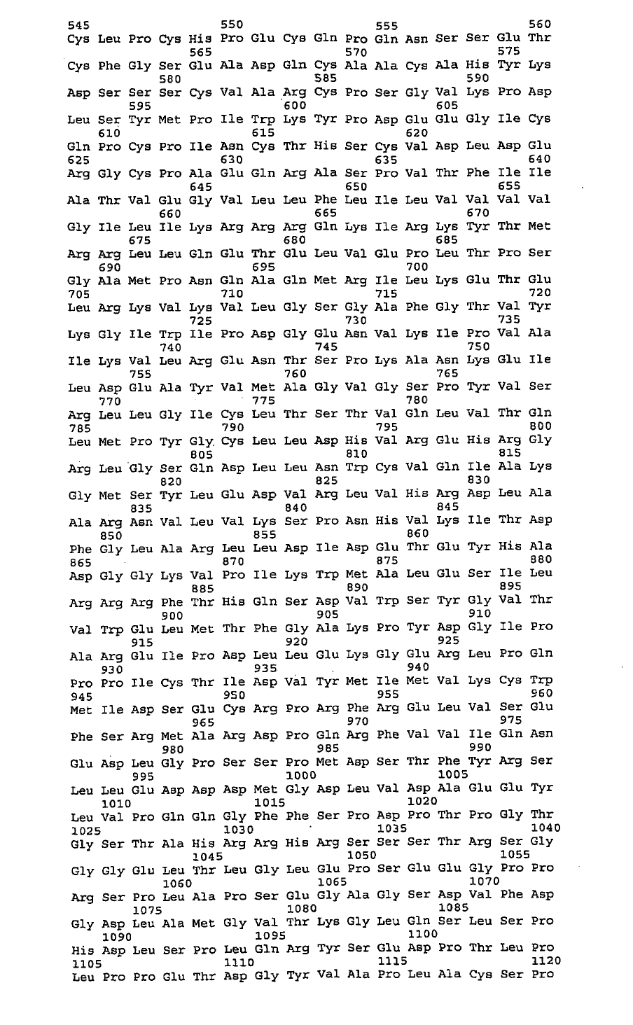

The ICD that may form part of the present invention may be of human, rat or mouse origin. Human ICD (FIG. 1; SEQ ID NO:12) inclusively spans the region of Lys 676 to Val 1255. The rat ICD is set forth in FIG. 2 and SEQ ID NO:13 as inclusively spanning the region of Lys 677 to Val 1256.

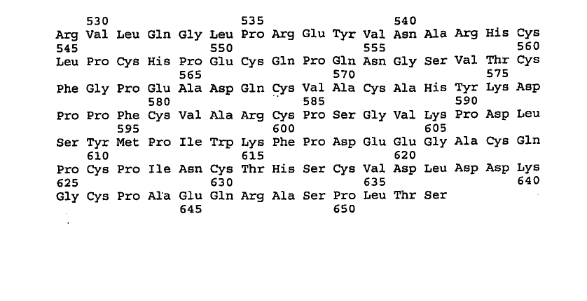

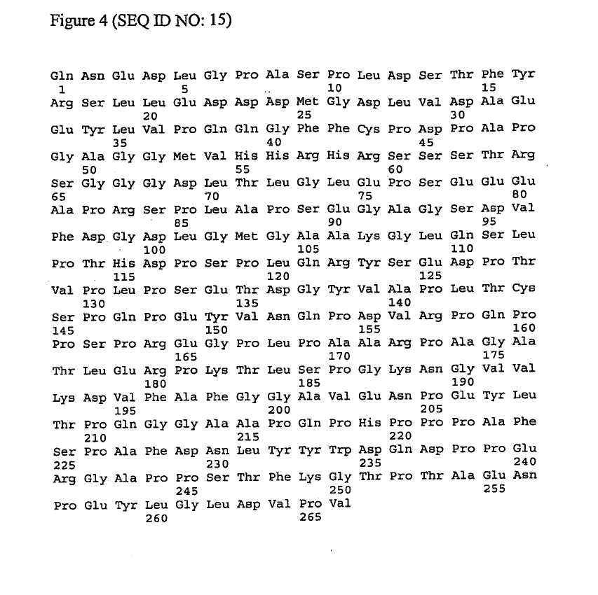

The PD that may form part of the present invention may be of human, rat or mouse origin. The human PD is set forth in FIG. 4 (amino acid sequence 1 to 266 of SEQ ID NO:15, equivalent to amino acid sequence 990 to 1255 of HER-2/neu). The human PD may be the human ΔPD, as shown in FIG. 15 (amino acid sequence 1 to 59 of SEQ ID NO:16, equivalent to amino acid sequence 990 to 1050 of HER-2/neu). The rat PD is shown in FIG. 2 and SEQ ID NO:13 as inclusively spanning the region of Gln 991 to Val 1256. The rat PD may be the rat ΔPD, which is shown in FIG. 2 and SEQ ID NO:13 as inclusively spanning the region of Gln 991 to Arg 1049.

In one embodiment, a human ECD can be fused with either (i) a human ICD or a rat ICD or (ii) a human PD or ΔPD, or a rat PD or ΔPD. In another embodiment, a rat ECD can be fused with either (i) a human ICD or a rat ICD or (ii) a human PD or ΔPD, or a rat PD or ΔPD.

The fusion protein that may form part of the present invention may comprise a HER-2/neu extracellular domain fused to a HER-2/neu phosphorylation domain. The protein may have a sequence at least 80%, 90% or 95% identical to the sequence of SEQ ID NO:17, or a sequence at least 80%, 90% or 95% identical to the sequence of SEQ ID NO:14 fused to a sequence at least 80%, 90% or 95% identical to the sequence of SEQ ID NO:15. Alternatively, the protein may comprise a sequence at least 80%, 90% or 95% identical to the sequence of SEQ ID NO:3 directly fused to an amino acid sequence at least 80%, 90% or 95% identical to the sequence inclusive of GIn 991 to Val 1256 of SEQ ID NO:13, or a sequence at least 80%, 90% or 95% identical to the sequence of SEQ ID NO:3 (17) fused to the amino acid sequence at least 80%, 90% or 95% identical to the sequence inclusive of Gln 991 to Val 1256 of SEQ ID NO:13. Alternatively, the protein may comprise a sequence at least 80%, 90% or 95% identical to the sequence of SEQ ID NO:19 directly fused to a sequence at least 80%, 90% or 95% identical to the sequence of SEQ ID NO:15, or a sequence at least 80%, 90% or 95% identical to the sequence of SEQ ID NO:19 fused to a sequence at least 80%, 90% or 95% identical to the sequence of SEQ ID NO:15. Alternatively, the protein may comprise a sequence at least 80%, 90% or 95% identical to the sequence of SEQ ID NO:19 directly fused to the amino acid sequence inclusive of Gln 991 to Val 1256 of SEQ ID NO:13, or a sequence at least 80%, 90% or 95% identical to the sequence of SEQ ID NO:19 fused to a sequence at least 80%, 90% or 95% identical to the amino acid sequence inclusive of Gln 991 to Val 1256 of SEQ ID NO:13.

In an alternative embodiment, the fusion protein may comprise a HER-2/neu extracellular domain fused to a fragment of the HER-2/neu phosphorylation domain. In one embodiment, the protein may have a sequence at least 80%, 90% or 95% identical to the sequence of SEQ ID NO:18, or a sequence at least 80%, 90% or 95% identical to the sequence of SEQ ID NO:14 fused to a sequence at least 80%, 90% or 95% identical to the sequence of SEQ ID NO:16. Alternatively, the protein may comprise a sequence at least 80%, 90% or 95% identical to the sequence of SEQ ID NO:14 directly fused to a sequence at least 80%, 90% or 95% identical to the amino acid sequence inclusive of Gln 991 to Arg 1049 of SEQ ID NO:2, or a sequence at least 80%, 90% or 95% identical to the sequence of SEQ ID NO:14 fused to a sequence at least 80%, 90% or 95% identical to the amino acid sequence inclusive of Gln 991 to Arg 1049 of SEQ ID NO:13. Alternatively, the protein may comprise a sequence at least 80%, 90% or 95% identical to the sequence of SEQ ID NO:19 directly fused to a sequence at least 80%, 90% or 95% identical to the sequence of SEQ ID NO:16, or a sequence at least 80%, 90% or 95% identical to the sequence of SEQ ID NO:19 fused to a sequence at least 80%, 90% or 95% identical to the sequence of SEQ ID NO:16. Alternatively, the protein may comprise a sequence at least 80%, 90% or 95% identical to the sequence of SEQ ID NO:19 directly fused to a sequence at least 80%, 90% or 95% identical to the amino acid sequence inclusive of Gln 991 to Arg 1049 of SEQ ID NO:13, or a sequence at least 80%, 90% or 95% identical to the sequence of SEQ ID NO:19 fused to a sequence at least 80%, 90% or 95% identical to the amino acid sequence inclusive of Gln 991 to Arg 1049 of SEQ ID NO:13.

In an alternative embodiment, the fusion protein may comprise a HER-2/neu extracellular domain fused to a HER-2/neu intracellular domain. Alternatively, the protein may comprise a sequence at least 80%, 90% or 95% identical to the sequence of SEQ ID NO:14 fused directly to a sequence at least 80%, 90% or 95% identical to the amino acid sequence inclusive of Lys 676 to Val 1255 in SEQ ID NO:12, or a sequence at least 80%, 90% or 95% identical to the sequence of SEQ ID NO:14 fused to a sequence at least 80%, 90% or 95% identical to the amino acid sequence inclusive of Lys 676 to Val 1255 of SEQ ID NO:12 via at least one of a chemical or amino acid linking group. Alternatively, the protein may comprise a sequence at least 80%, 90% or 95% identical to the sequence of SEQ ID NO:14 directly fused to a sequence at least 80%, 90% or 95% identical to the amino acid sequence inclusive of Lys 677 to Val 1256 of SEQ ID NO:13, or wherein the protein comprises a sequence at least 80%, 90% or 95% identical to the sequence of SEQ ID NO:14 fused to a sequence at least 80%, 90% or 95% identical to the amino acid sequence inclusive of Lys 677 to Val 1256 of SEQ ID NO:2 via at least one of a chemical or amino acid linking group. Alternatively, the protein may comprise a sequence at least 80%, 90% or 95% identical to the sequence of SEQ ID NO:19 directly fused to a sequence at least 80%, 90% or 95% identical to the amino acid sequence inclusive of Lys 676 to Val 1255 of SEQ ID NO:12, or a sequence at least 80%, 90% or 95% identical to the sequence of SEQ ID NO:19 fused to a sequence at least 80%, 90% or 95% identical to the amino acid sequence inclusive of Lys 676 to Val 1255 of SEQ ID NO:12 via at least one of a chemical or amino acid linking group. Alternatively, the protein may comprise a sequence at least 80%, 90% or 95% identical to the sequence of SEQ ID NO:19 directly fused to a sequence at least 80%, 90% or 95% identical to the amino acid sequence inclusive of Lys 677 to Val 1256 of SEQ ID NO:13, or a sequence at least 80%, 90% or 95% identical to the sequence of SEQ ID NO:19 fused to a sequence at least 80%, 90% or 95% identical to the amino acid sequence inclusive of Lys 677 to Val 1256 of SEQ ID NO:13 via at least one of a chemical or amino acid linking group.

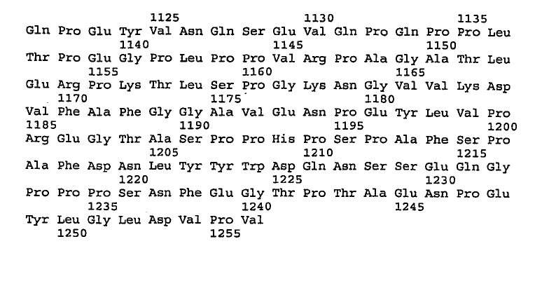

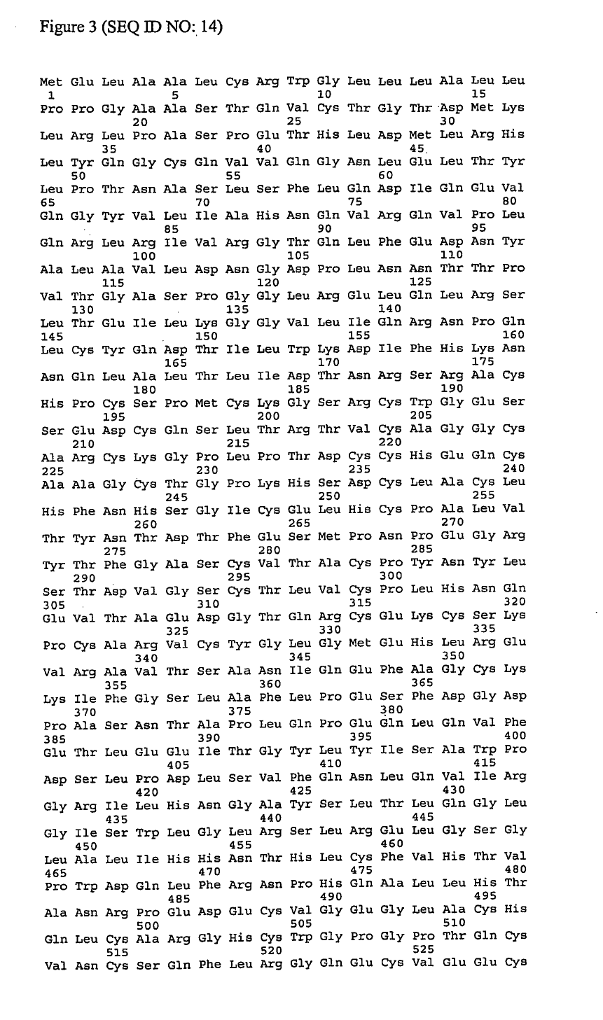

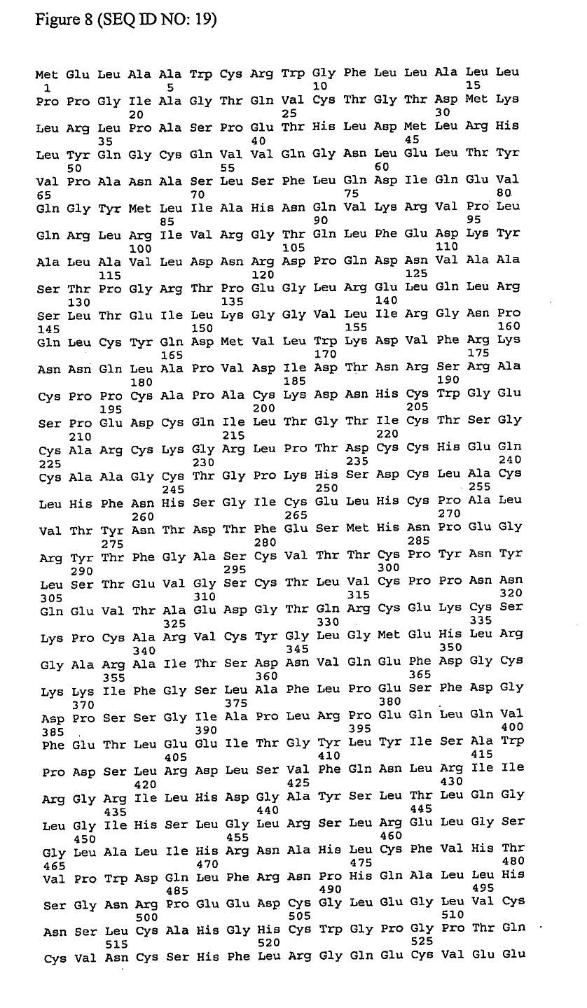

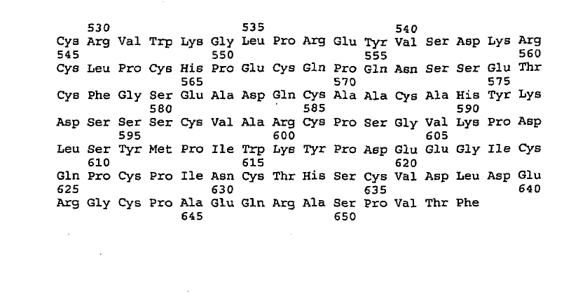

The ECD-ICD fusion proteins which may be used in the present invention, which will be understood to include variants, include any possible combination between human and non-human polypeptides. Non-human polypeptides comprise polypeptides from any mammal, such as, e.g., rat, mouse, guinea pig, horse, cow, pig, sheep, dog, etc. In one embodiment, the ECD-ICD fusion proteins include:
(i) human ECD—human ICD fusion proteins, such as those formed by linking the human ECD of FIG. 3 (SEQ ID NO:14) with the human ICD, which is the amino acid sequence inclusively spanning Lys 676 to Val 1255, as shown in FIG. 1 (SEQ ID NO:12), with or without a chemical and/or amino acid linking group, and variants thereof;
(ii) (ii) rat ECD—rat ICD fusion proteins, such as those formed by linking the rat ECD of FIG. 8 (SEQ ID NO:19) with the rat ICD, which is the amino acid sequence inclusively spanning Lys 677 to Val 1256, as shown in FIG. 2 (SEQ ID NO:13), with or without a chemical and/or amino acid linking group, and variants thereof;
(iii) human ECD—rat ICD fusion proteins, such as those formed by linking the human ECD shown in FIG. 3 (SEQ ID NO:14) with the rat ICD, which is the amino acid sequence inclusively spanning Lys 677 to Val 1256, as shown in FIG. 2 (SEQ ID NO:13), with or without a chemical and/or amino acid linking group, and variants thereof; and
(iv) rat ECD—human ICD fusion proteins, such as those formed by linking the rat ECD, as shown in FIG. 8 (SEQ ID NO:19), with the human ICD, which is the amino acid sequence inclusively spanning Lys 676 to Val 1255, as shown in FIG. 1 (SEQ ID NO:12), with or without a chemical and/or amino acid linking group, and variants thereof.

Any variants of the ECD-ICD fusion proteins described herein are included as fusion proteins of the present invention. In one embodiment, such variants are substantially identical or substantially similar to the native HER-2/neu ECD-ICD protein and retain the ability to stimulate an immune response.

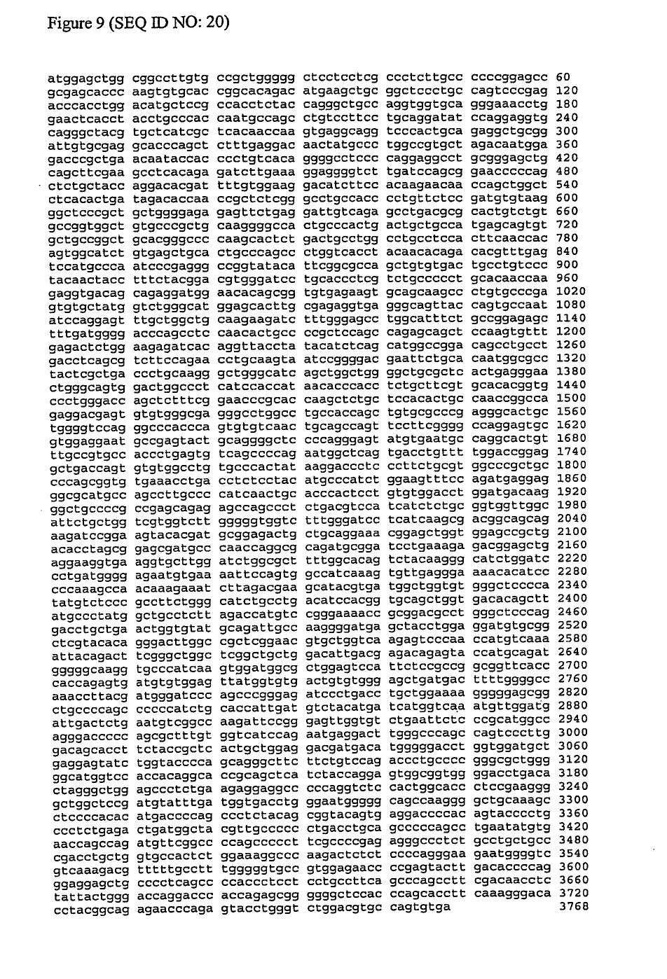

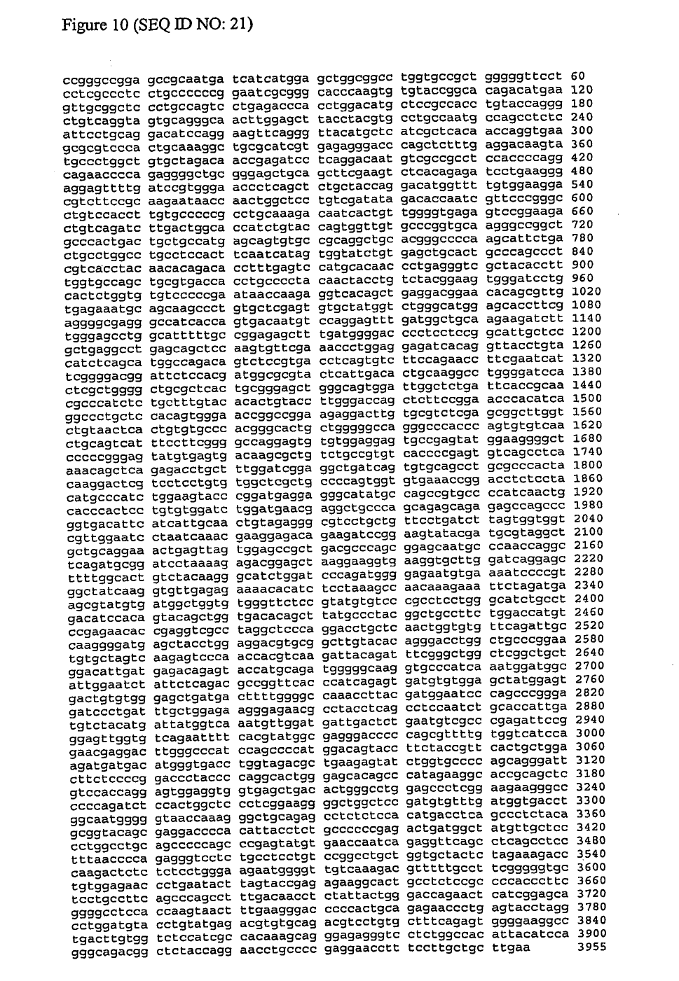

Human DNA sequences that encode the ECD protein are shown, for example, in FIG. 9 (SEQ ID NO:20) as inclusively spanning nucleotide 1 to nucleotide 1959. Human DNA sequences that encode the ICD protein are shown, for example, in FIG. 9 (SEQ ID NO:20) as inclusively spanning nucleotide 2026 to nucleotide 3765. The effect of any sequence modification on the ability of a HER-2/neu ECD-ICD protein to produce an immune response may be readily determined, for example, by analyzing the ability of the mutated HER-2/neu ECD-ICD protein to induce a T cell response using, for example, the methods described herein, or by analyzing the ability of the mutated HER-2/neu ECD-ICD protein to produce antibodies.

The ECD-PD fusion proteins which may be used in the present invention, which will be understood to include variants, include any possible combination between human and non-human polypeptides. Non-human polypeptides comprise, e.g., rat, mouse, guinea pig, horse, cow, pig, sheep, dog, etc. In one embodiment, the ECD-PD fusion proteins include:

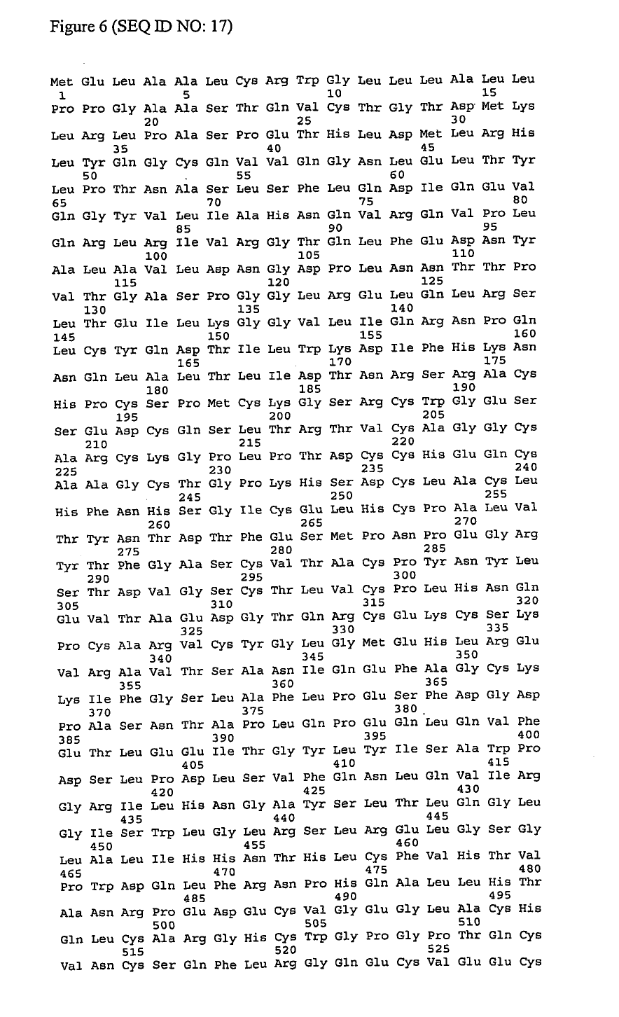

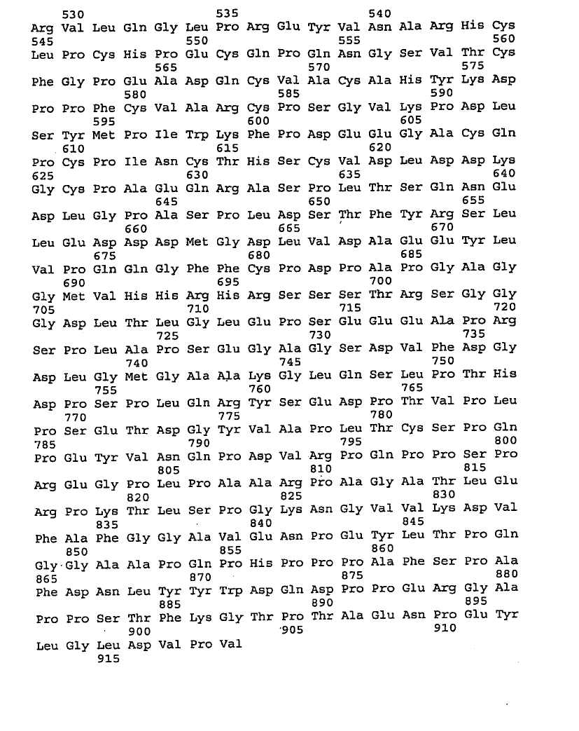

(i) human ECD—human PD fusion proteins, such as shown in FIG. 6 (SEQ ID NO:17) and variants thereof, including fusion proteins formed by linking the human ECD of FIG. 3 (SEQ ID NO:14) with the human PD of FIG. 4 (SEQ ID NO:15) with or without a chemical and/or amino acid linking group, and variants thereof;
(ii) rat ECD—rat PD fusion proteins, such as those formed by linking the rat ECD of FIG. 8 (SEQ ID NO:19) with the rat PD, which is the amino acid sequence inclusively spanning Gln 991 to Val 1256, as shown in FIG. 2 (SEQ ID NO:13), with or without a chemical and/or amino acid linking group, and variants thereof;
(iii) human ECD—rat PD fusion proteins, such as those formed by linking the human ECD shown in FIG. 3 (SEQ ID NO:14) with the rat PD, which is the amino acid sequence inclusively spanning Gln 991 to Val 1256, as shown in FIG. 2 (SEQ ID NO:13), with or without a chemical and/or amino acid linking group, and variants thereof; and
(iv) rat ECD—human PD fusion proteins, such as those formed by linking the rat ECD, as shown in FIG. 8 (SEQ ID NO:19), with the human PD, as shown in FIG. 4 (SEQ ID NO:15), with or without a chemical and/or amino acid linking group, and variants thereof.

Any variants of the ECD-PD fusion proteins are included as embodiments of the present invention. In one embodiment, such variants are substantially identical or substantially similar to the native HER-2/neu ECD-PD protein and retain the ability to stimulate an immune response. Human DNA sequences that encode the ECD protein are shown, for example, in FIG. 9 (SEQ ID NO:20) as inclusively spanning nucleotide 1 to nucleotide 1959. Human DNA sequences that encode the PD protein are shown, for example, in FIG. 9 (SEQ ID NO:20) as inclusively spanning nucleotide 2968 to nucleotide 3765. The effect of any sequence modification on the ability of a HER-2/neu ECD-PD protein to produce an immune response may be readily determined, for example, by analyzing the ability of the mutated HER-2/neu ECD-PD protein to induce a T cell response using, for example, the methods described herein, or by analyzing the ability of the mutated HER-2/neu ECD-PD protein to produce antibodies.

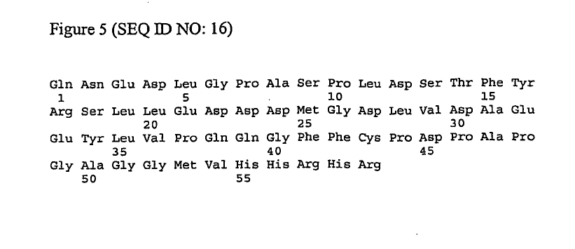

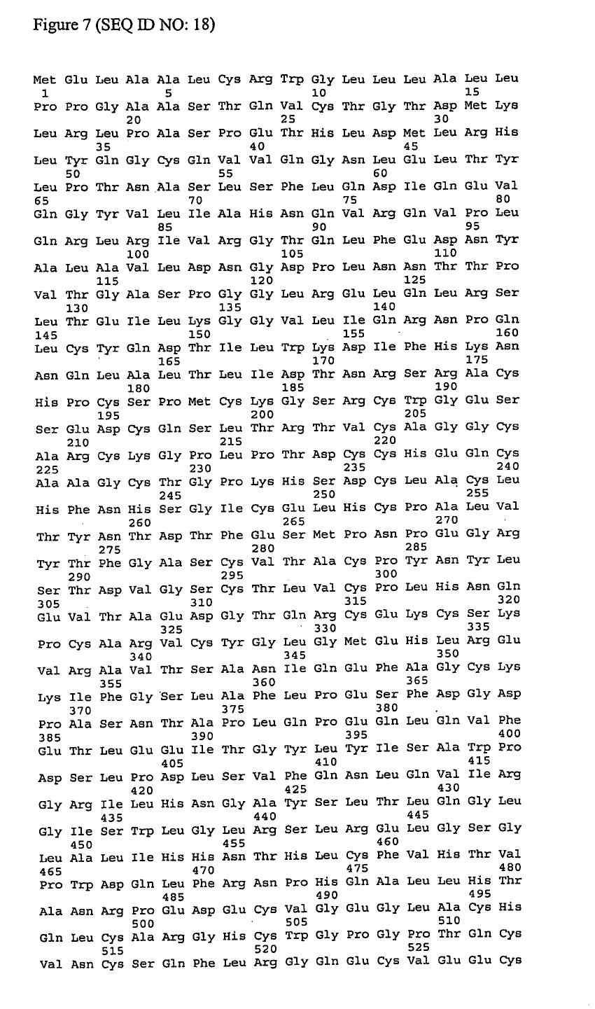

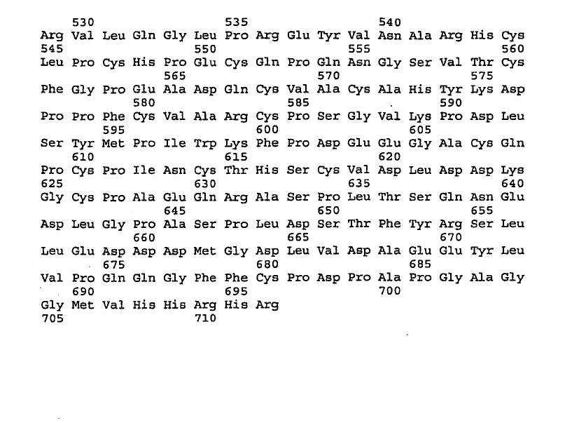

In another embodiment, the ECD-PD fusion proteins are ECD-ΔPD fusion proteins of the present invention, which will be understood to include variants, including any possible combination between human and non-human polypeptides. Non-human polypeptides comprise, e.g., rat, mouse, guinea pig, horse, cow, pig, sheep, dog, etc. In one embodiment, the ECD-ΔPD fusion proteins include:
(i) human ECD—human ΔPD fusion proteins, such as shown in FIG. 7 (SEQ ID NO:18) and variants thereof, including fusion proteins formed by linking the human ECD of FIG. 3 (SEQ ID NO:14) with the human ΔPD of FIG. 5 (SEQ ID NO:16) with or without a chemical and/or amino acid linking group, and variants thereof;
(ii) rat ECD—rat ΔPD fusion proteins, such as those formed by linking the rat ECD of FIG. 8 (SEQ ID NO:19) with the rat ΔPD, which is the amino acid sequence inclusively spanning Gln 991 to Arg 1049, as shown in FIG. 2 (SEQ ID NO:13), with or without a chemical and/or amino acid linking group, and variants thereof;
(iii) human ECD—rat ΔPD fusion proteins, such as those formed by linking the human ECD shown in FIG. 3 (SEQ ID NO:14) with the rat ΔPD, which is the amino acid sequence inclusively spanning Gln 991 to Arg 1049, as shown in FIG. 2 (SEQ ID NO:13), with or without a chemical and/or amino acid linking group, and variants thereof; and
(iv) rat ECD—human ΔPD fusion proteins, such as those formed by linking the rat ECD, as shown in FIG. 8 (SEQ ID NO: 19), with the human ΔPD, as shown in FIG. 5 (SEQ ID NO:16), with or without a chemical and/or amino acid linking group, and variants thereof.

Any variants of the ECD-ΔPD fusion proteins are included as embodiments of the present invention. In one embodiment, such variants are substantially identical or substantially similar to the native HER-2/neu ECD-ΔPD protein and retain the ability to stimulate an immune response. Human DNA sequences that encode the ECD protein are shown, for example, in FIG. 9 (SEQ ID NO:20) as inclusively spanning nucleotide 1 to nucleotide 1959. Human DNA sequences that encode the ΔPD protein of SEQ ID NO:16 are shown, for example, in FIG. 9 (SEQ ID NO:20) as inclusively spanning nucleotide 2968 to nucleotide 3144. The effect of any sequence modification on the ability of a HER-2/neu ECD-ΔPD protein to produce an immune response may be readily determined, for example, by analyzing the ability of the mutated HER-2/neu ECD-ΔPD protein to induce a T cell response using, for example, the methods described herein, or by analyzing the ability of the mutated HER-2/neu ECD-ΔPD protein to produce antibodies.

In one embodiment, immunogenic component (b) comprises an ECD-PD fusion protein or a polynucleotide encoding such a fusion protein.

In an embodiment of the present invention in which component (b) is a protein, component (b) may further comprise adjuvant or immunostimulant such as but not limited to one capable of stimulating a TH1 type response. In one embodiment of the present invention, component (b) comprises an adjuvant capable of stimulating a TH1 type response.

### Adjuvants Suitable for Protein Formulations

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p 407-419) and has the following structure:

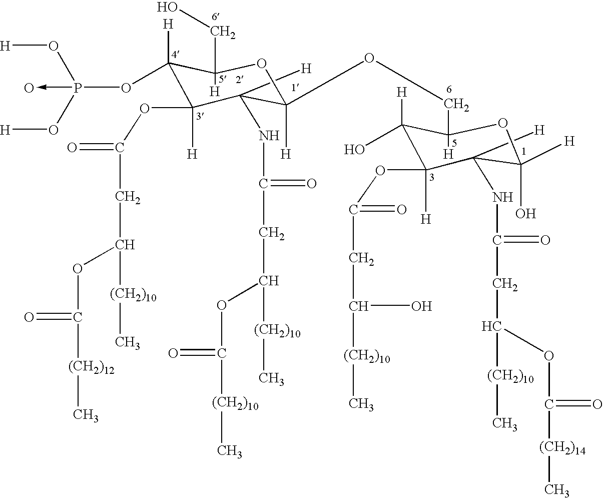

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof. In one embodiment, the immunogenic composition comprises 3D-MPL.

In one embodiment the form of 3D-MPL which may be used is in the form of an emulsion having a small particle size less than 0.2 μm in diameter, and its method of manufacture is disclosed in WO 94/21292, incorporated herein by reference. Aqueous formulations comprising monophosphoryl lipid A and a surfactant have been described in WO9843670, incorporated herein by reference.

The bacterial lipopolysaccharide derived adjuvants to be formulated in the compositions of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (Hilgers et al., 1986, Int. Arch. Allergy. Immunol., 79(4):392-6; Hilgers et al., 1987, Immunology, 60(1):141-6; and EP 0 549 074 B1). In one embodiment the bacterial lipopolysaccharide adjuvant is 3D-MPL.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

3D-MPL is an agonist of Toll-like receptor 4 (TLR-4). In one embodiment, one or more TLR-4 agonist(s) may be included in an immunogenic composition of the present invention. TLR-4 agonists include: lipopolysaccharide (LPS) from gram-negative bacteria, or fragments thereof; heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2 and MPL, for example 3D-MPL.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739, incorporated herein by reference.). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Other saponins which have been used in systemic vaccination studies include those derived from other plant species such as Gypsophila and Saponaria (Bomford et al., Vaccine, 10(9):572-577, 1992).

One adjuvant system which may be used in the present invention comprises a non-toxic lipid A derivative and a saponin derivative. A particular adjuvant system which may be used comprises 3D-MPL and QS21, as disclosed in, for example, WO 94/00153, incorporated herein by reference. A further system which may be used comprises 3D-MPL and QS21, in which the QS21 is quenched with cholesterol, as disclosed in, for example, WO 96/33739, incorporated herein by reference.

In an alternative embodiment of the present invention, component (b) additionally comprises an adjuvant composition comprising a saponin, together with an immunostimulatory oligonucleotide. For example, the adjuvant composition may comprise QS21, together with immunomodulatory oligonucleotide, for example oligonucleotide containing unmethylated CG motives (CpG oligonucleotide).

In one embodiment of the present invention, an immunostimulatory oligonucleotide which may be included in an adjuvant is selected from the group:—

SEQ ID No 22
TCC ATG ACG TTC CTG ACG TT (CpG 1826)
SEQ ID No 23
TCT CCC AGC GTG CGC CAT (CpG 1758)
SEQ ID No 24
ACC GAT GAC GTC GCC GGT GAC GGC ACC ACG
SEQ ID No 25
TCG TCG TTT TGT CGT TTT GTC GTT (CpG 2006)
SEQ ID No 26
TCC ATG ACG TTC CTG ATG CT (CpG 1668)

In an alternative embodiment of the present invention, component (b) additionally comprises an adjuvant composition comprising an oil-in-water emulsion, for example as described in EP 382 271, incorporated herein by reference.

In a further embodiment, component (b) additionally comprises an adjuvant composition formulated with 3D-MPL, QS21 and CpG oligonucleotide together with a liposome or oil-in-water emulsion carrier, for example as described in WO02/32450, incorporated herein by reference. Such formulations may produce both a humoral and cellular mediated response. In comparisons with adjuvant formulation comprising just QS21 and 3D-MPL, the formulation of the invention may adduce, in mice, advantageously a stronger TH1 response.

In a yet further embodiment of the present invention, the adjuvant is SB62′c, an adjuvant comprising an oil-in-water emulsion and a saponin, wherein the oil is a metabolisable oil, and the ratio of the metabolisable oil:saponin (w/w) is in the range of 1:1 to 200:1 (oil-in-water emulsion low dose) described in WO99/11241, the full teaching of which is incorporated herein by reference. In one embodiment, the ratio of the metabolisable oil:saponin (w/w) is substantially 48:1. The saponin may be a QuilA, such as QS21. In one example, the metabolisable oil is squalene. The SB62′c adjuvant composition may further comprise a sterol, for example cholesterol. The SB62′c adjuvant composition may additionally or alternatively further comprise one or more immunomodulators, for example: 3D-MPL and/or α-tocopherol. In an embodiment of SB62′c which comprises 3D-MPL, the ratio of QS21:3D-MPL (w/w) maybe from 1:10 to 10:1, for example 1:1 to 1:2.5, or 1:1 to 1:20.

Thus, in one embodiment of the adjuvant SB62′c, the ratio of the metabolisable oil:saponin (w/w) is in the range of 1:1 to 200:1 or is substantially 48:1, the saponin is QS21 and the adjuvant also includes 3D-MPL (oil-in-water emulsion low dose, QS21, 3D-MPL).

In a further embodiment of the present invention, the adjuvant consists of an oil-in-water emulsion comprising a tocol, for example as described in EP0382271, incorporated herein by reference. In a further embodiment, the oil-in-water emulsion which may be used comprises α-tocopherol.

In one embodiment, the adjuvant is an adjuvant composition as described herein, presented within a liposome, for example as described in EP822831, incorporated herein by reference. In a further embodiment, the adjuvant may comprise montanide ISA51.

### Vaccine

The present invention also provides a vaccine comprising an immunogenic composition as described herein, with a pharmaceutically acceptable excipient, adjuvant or vehicle. The present invention also provides a process for the manufacture of a vaccine composition comprising mixing an immunogenic composition as described herein with appropriate pharmaceutically acceptable vehicles, adjuvants or excipients. Appropriate vehicles and excipients are well known in the art and include for example water or buffers. Vaccine preparation is generally described in Vaccine Design (“The subunit and adjuvant approach” (eds Powell M. F. & Newman M. J.) (1995) Plenum Press New York).

### Polynucleotide

In one embodiment of the present invention, component (b) comprises a polynucleotide sequence which encodes the fusion protein of the present invention as described herein.

In a further embodiment, the polynucleotide sequence may hybridise under stringent conditions to a polynucleotide sequence encoding the fusion protein. As herein used, the terms “stringent conditions” and “stringent hybridization conditions” mean hybridization occurring only if there is at least 95% and preferably at least 97% identity between the sequences. A specific example of stringent hybridization conditions is overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 micrograms/ml of denatured, sheared salmon sperm DNA, followed by washing the hybridization support in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein.

The polynucleotide sequences encoding the fusion polypeptide therefore include conservatively modified variants, polymorphic variants, alleles, mutants, sub-sequences, and interspecies homologues. In an embodiment in which component (b) comprises a polynucleotide, component (b) may additionally comprise an adjuvant, or be administered either concomitantly with or sequentially with an adjuvant or immunostimulatory agent.

The polynucleotide may be presented within any of a variety of delivery systems known to those of ordinary skill in the art, including nucleic acid expression systems, bacteria and viral expression systems. Numerous gene delivery techniques are well known in the art, such as those described by Rolland (1998) Crit. Rev. Therap. Drug Carrier Systems 15:143-198, and references cited therein. Appropriate nucleic acid expression systems contain the necessary DNA sequences for expression in the patient (such as a suitable promoter and terminating signal). Bacterial delivery systems involve the administration of a bacterium (such as Bacillus Calmette-Guerin) that expresses an immunogenic portion of the fusion protein on its cell surface or secretes such an epitope. In one embodiment, the DNA may be introduced using a viral expression system or vector (e.g., vaccinia, pox virus, retrovirus, or adenovirus), which may involve the use of a non-pathogenic (defective), replication competent virus. Suitable systems are disclosed, for example, in Pisher-Hoch et al. (1989) Proc. Natl. Acad. Sci. USA 86:317-321;Flexner et al. (1989) Ann. N.Y. Acad. Sci. 569:86-103; Flexner et al. (1990) Vaccine 8:17-21; U.S. Pat. Nos. 4,603,112, 4,769,330, 4,777,127 and 5,017,487; WO 89/01973; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502;Guzman et al. (1993) Circulation 88:2838-2848; and Guzman et al. (1993) Cir. Res. 73:1202-1207. Techniques for incorporating DNA into such expression systems are well known to those of ordinary skill in the art. The DNA may also be “naked,” as described, for example, in Ulmer et al. (1993) Science 259:1745-1749 and reviewed by Cohen (1993) Science 259:1691-1692. The uptake of naked DNA maybe increased by coating the DNA onto biodegradable beads, which are efficiently transported into the cells.

It will be apparent that a vaccine or immunogenic composition of the present invention may comprise both a polynucleotide and a polypeptide component. Such vaccines or immunogenic compositions may provide for an enhanced immune response.

Immunostimulatory agents which may be used with a polynucleotide sequence include synthetic imidazoquinolines such as imiquimod [S-26308, R-837] or any other molecule known to stimulate Toll-like receptor 7, (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules as either protein or peptide, this would include pro-inflammatory cytokines such as Interferons, particular interferons and GM-CSF, IL-1 alpha, IL-1 beta, TGF-alpha and TGF -beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A. Other bacterial derived immunostimulating proteins include, Cholera Toxin, E.Coli Toxin and mutant toxoids thereof. Examples of adjuvants for eliciting a predominantly Th1-type response include, for example, a Lipid A derivative such as monophosphoryl lipid A, or. 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another adjuvant which may be used comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

In one embodiment of the present invention, an immunostimulatory oligonucleotide which may be included in an adjuvant is selected from the group:—

SEQ ID No 22
TCC ATG ACG TTC CTG ACG TT (CpG 1826)
SEQ ID No 23
TCT CCC AGC GTG CGC CAT (CpG 1758)
SEQ ID No 24
ACC GAT GAC GTC GCC GGT GAC GGC ACC ACG
SEQ ID No 25
TCG TCG TTT TGT CGT TTT GTC GTT (CpG 2006)
SEQ ID No 26
TCC ATG ACG TTC CTG ATG CT (CpG 1668)

### Combinations

Component (a) and component (b) may be employed in combination concomitantly or sequentially in any therapeutically appropriate combination.

The combination may be employed in accordance with the invention by concomitant administration of components (a) and (b), for example by (1) administering a unitary pharmaceutical composition comprising both components or (2) concomitantly administering to a subject separate pharmaceutical compositions each comprising one of the components.

In the embodiment in which components (a) and (b) are administered separately, the combination may be administered at the same time or in a sequential manner in which one is administered first and the other second or vice versa.

As used herein, sequential administration refers to administration of both components (a) and (b) within a biologically relevant time frame. Examples of sequential administration include, e.g., administration of the second component as soon as administration of the first is completed; or administering the second component at a time when the subject is experiencing the biologic effects of the first-administered component. Thus it will be apparent that when component (b) is administered first, component (a) is administered during the period of an immune response, for example an antibody and/or T-cell response elicited by component (b). In one embodiment of the present invention in which component (b) is given in a prime-boost regimen, component (a) may be given concomitant with a “boost” administration of component (b).

Where component (a) is administered first, component (b) is administered during the time period when component (a) is present in the subject's body. It will be apparent to those skilled in the art that the components may be administered in other than a 1:1 manner, e.g., component (b) may be administered more than once, in advance of administration of component (a); administration of component (b) may be followed by multiple administrations of component (a) within the biologically relevant time frame, etc.

In one embodiment, component (a) is given in advance of component (b). Component (a) may be given once or more than once in advance of component (b).

In another embodiment, component (b) is given in advance of component (a). Component (b) may be given once or more than once in advance of component (a). In one embodiment, component (a) may be administered to an individual, wherein the individual has been previously administered with immunogenic composition (b).

It is understood that if component (b) is given more than once, one or more administration may be protein and one or more administration may be DNA.

The present invention may also include administration of at least one additional cancer treatment therapy in combination concomitantly or sequentially in any therapeutically appropriate combination with the combinations of the present invention. The additional cancer treatment therapy may include radiation therapy, surgical therapy and/or at least one additional chemotherapeutic therapy comprising administration of at least one additional anti-neoplastic agent.

In an embodiment of the present invention in which component (b) is administered in advance of component (a), component (b) may be given at a time sufficient in advance of component (a) to allow the generation of an immune response, for example a polyclonal antibody response. In one embodiment of the present invention, component (a) may be given at the peak of an immune response generated against component (b). In one embodiment, component (a) may be given at the peak of anamnestic (memory) response.

### Administration of Component (a)

While it is possible that, for use in therapy, compounds of formula I, II, III, IV as well as salts, solvates and physiologically function derivatives thereof of component (a) may be administered as the raw chemical, it is possible to present the active ingredient as a pharmaceutical composition. Accordingly, in one embodiment, component (a) of the invention is provided as a pharmaceutical composition comprising compounds of formula I, II, III and/or IV, and salts, solvates, and physiologically functional derivatives thereof, and one or more pharmaceutically acceptable carriers, diluents, or excipients.

The carrier(s), diluent(s) or excipient(s) must be acceptable in the sense of being compatible with the other ingredients of the formulation and not deleterious to the recipient thereof.

According to another aspect of the invention there is also provided a process for the preparation of a pharmaceutical formulation of component (a) comprising admixing a compound of the formula I, II, III and/or IV, and/or salts, solvates, and/or physiologically functional derivatives thereof, with one or more pharmaceutically acceptable carriers, diluents or excipients and providing such a pharmaceutical formulation in combination with component (b) of the present invention.

The components of the pharmaceutical compositions of component (a) of the present invention, may be formulated for administration by any route, and the appropriate route will depend on the specific cancer being treated as well as the subjects to be treated. Suitable pharmaceutical formulations include those for oral, rectal, nasal, topical (including buccal, sub-lingual, and transdermal), vaginal or parenteral (including intramuscular, sub-cutaneous, intravenous, and directly into the affected tissue) administration or in a form suitable for administration by inhalation or insufflation. The formulations may, where appropriate, be conveniently presented in discrete dosage units and may be prepared by any of the methods well know in the pharmacy art.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agents can also be present.

Capsules are made by preparing a powder mixture as described above, and filling formed gelatine sheaths. Glidants and lubricants such as colloidal silica, talc, magnesium stearate, calcium stearate or solid polyethylene glycol can be added to the powder mixture before the filling operation. A disintegrating or solubilising agent such as agar-agar, calcium carbonate or sodium carbonate can also be added to improve the availability of the medicament when the capsule is ingested.

Moreover, when desired or necessary, suitable binders, lubricants, disintegrating agents and colouring agents can also be incorporated into the mixture. Suitable binders include starch, gelatine, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methylcellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilisers and emulsifiers such as ethoxylated isostearyl alcohols and polyoxy ethylene sorbitol ethers, preservatives, flavour additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The components of the pharmaceutical compositions of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The components of the pharmaceutical compositions of the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouthwashes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of component (a) of the pharmaceutical compositions of the present invention will depend on a number of factors including, but not limited to, the age and weight of the mammal, the precise disorder requiring treatment and its severity, the nature of the formulation, and the route of administration, and will ultimately be at the discretion of the attendant physician or veterinarian. Typically, the components of the pharmaceutical compositions of the present invention will be given for treatment in the range of 0.1 to 100 mg/kg body weight of recipient (mammal) per day and more usually in the range of 1 to 10 mg/kg body weight per day. Acceptable daily dosages, may be from about 0.1 to about 1000 mg/day, for example from about 0.1 to about 100 mg/day.

### Administration of Component (b)

Routes and frequency of administration of the component (b) described herein, as well as dosage, will vary from individual to individual, and may be readily established using standard techniques. In general, the immunogenic compositions and/or vaccines may be administered by injection (e.g., intracutaneous, intramuscular, intravenous or subcutaneous), intranasally (e.g., by aspiration) or orally. In one embodiment, between 1 and 10 doses may be administered over a 52 week period. 6 doses may be administered, at intervals of 1 month, and booster vaccinations may be given periodically thereafter. Alternate protocols may be appropriate for individual patients. A suitable dose is an amount of a compound that, when administered as described above, is capable of promoting an anti-tumour immune response, and is at least 10-50% above the basal (i.e., untreated) level. For a vaccine, such response may be monitored by measuring the anti-tumour antibodies in a patient or by vaccine-dependent generation of cytolytic effector cells capable of killing the patient's tumour cells in vitro. Such vaccines may also be capable of causing an immune response that leads to an improved clinical outcome (e.g., more frequent remissions, complete or partial or longer disease-free survival) in vaccinated patients as compared to non-vaccinated patients.

In general, for immunogenic compositions and vaccines, the amount of each immunogen present in a dose ranges from about 1 μg to 5 mg, for example 100 μg to 5 mg, or for example 5 μg to 250 μg per kg of host. Suitable dose sizes will vary with the size of the patient, but will typically range from about 0.1 ml to about 5 ml.

In one embodiment, an initial or primary immunization will be made with a HER-2/neu immunogenic composition as described herein, for example a HER-2/neu fusion protein having, e.g., at least one of an ECD and/or a ICD or PD, and a subsequent or booster immunization will also be made. ECD-ICD and/or ECD-PD fusion proteins suitable for immunization include those described herein. It will be appreciated by one skilled in the art that where the HER-2/neu immunogenic composition is a fusion protein, the present invention contemplates the use of an intact HER-2/neu fusion protein as well as division of the Her-2/neu fusion protein into a plurality of peptides.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Generation of immune responses or increase of pre-existing immune responses to a HER-2/neu protein or fusion protein may also indicate use of a sufficient amount of component (b). Increases in pre-existing immune responses may correlate with an improved clinical outcome. Such immune responses may generally be evaluated using standard proliferation, cytotoxicity or cytokine assays, which may be performed using samples obtained from a patient before and after treatment.

### T Cells

Component (b) of the invention may also, or alternatively, comprise T cells specific for a polypeptide or fusion protein as described herein. Such cells may generally be prepared in vitro or ex vivo, using standard procedures. For example, T cells may be isolated from bone marrow, peripheral blood or a fraction of bone marrow or peripheral blood of a patient, using a commercially available cell separation system (see also U.S. Pat. Nos. 5,240,856 and 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide and/or an antigen presenting cell (APC) that expresses such a polypeptide. Such stimulation is performed under conditions and for a time sufficient to permit the generation of T cells that are specific for the fusion polypeptide. The polypeptide or polynucleotide may be present within a delivery vehicle, such as a microsphere, to facilitate the generation of specific T cells.

T cells are considered to be specific for a HER-2/neu fusion polypeptide if the T cells kill target cells coated with the fusion polypeptide or expressing a polynucleotide encoding the fusion polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al. (1994) Cancer Res. 54:1065-1070. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a HER-2/neu fusion polypeptide (100 ng/ml-100 μg/ml, for example 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFNγ) is indicative of T cell activation (see Coligan et al., Current Protocols in Immunology, vol. 1, Wiley Interscience (Greene 1998)). T cells that have been activated in response to a HER-2/neu fusion polypeptide, polynucleotide or fusion polypeptide-expressing APC may be CD4+ and/or CD8+. HER-2/neu -specific T cells may be expanded using standard techniques. Within some embodiments, the T cells are derived from a patient, or from a related or unrelated donor, and are administered to the patient following stimulation and expansion.

For therapeutic purposes, CD4+ or CD8+T cells that proliferate in response to a HER-2/neu polypeptide, polynucleotide or APC can be expanded in number either in vitro or in vivo. Proliferation of such T cells in vitro may be accomplished in a variety of ways. For example, the T cells can be re-exposed to a HER-2/neu polypeptide with or without the addition of T cell growth factors, such as interleukin-2, and/or stimulator cells that synthesize a HER-2/neu polypeptide. Alternatively, one or more T cells that proliferate in the presence of a HER-2/neu protein can be expanded in number by cloning. Methods for cloning cells are well known in the art, and include limiting dilution. Following expansion, the cells may be administered back to the patient as described, for example, by Chang et al. (1996) Crit. Rev. Oncol. Hematol. 22:213.

### Dendritic Cells

Component (b) of the invention may also, or alternatively, comprise dendritic cells (DCs) specific for a polypeptide or fusion protein as described herein. Such cells may generally be prepared in vitro or ex vivo, using standard procedures. An example of a method which may be used is described in WO01/74855, incorporated herein by reference.

In one embodiment, the mammal in the methods and uses of the present invention is a human.

### Example of a Method of Preparation of Component (a)

The free base, HCl salts, and ditosylate salts of the compound of Formula (I) may be prepared according to the procedures of International Patent Application No. PCT/EP99/00048, filed Jan. 8, 1999, and published as WO 99/35146 on Jul. 15, 1999, referred to above and International Patent Application No. PCT/US01/20706, filed Jun. 28, 2001 and published as WO 02/02552 on Jan. 10, 2002 and according to the appropriate Examples recited below. One such procedure for preparing the ditosylate salt of the compound of formula (I) is presented following in Scheme 1.

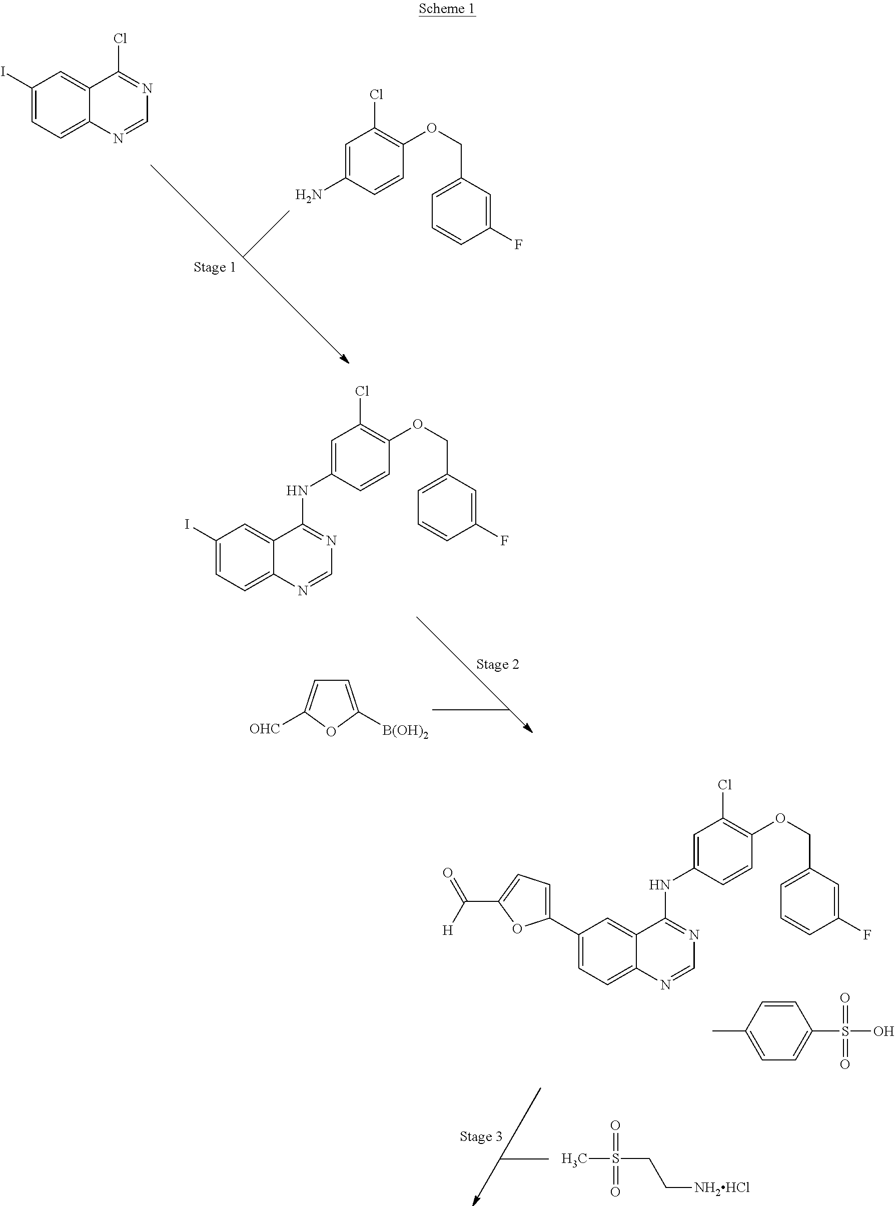

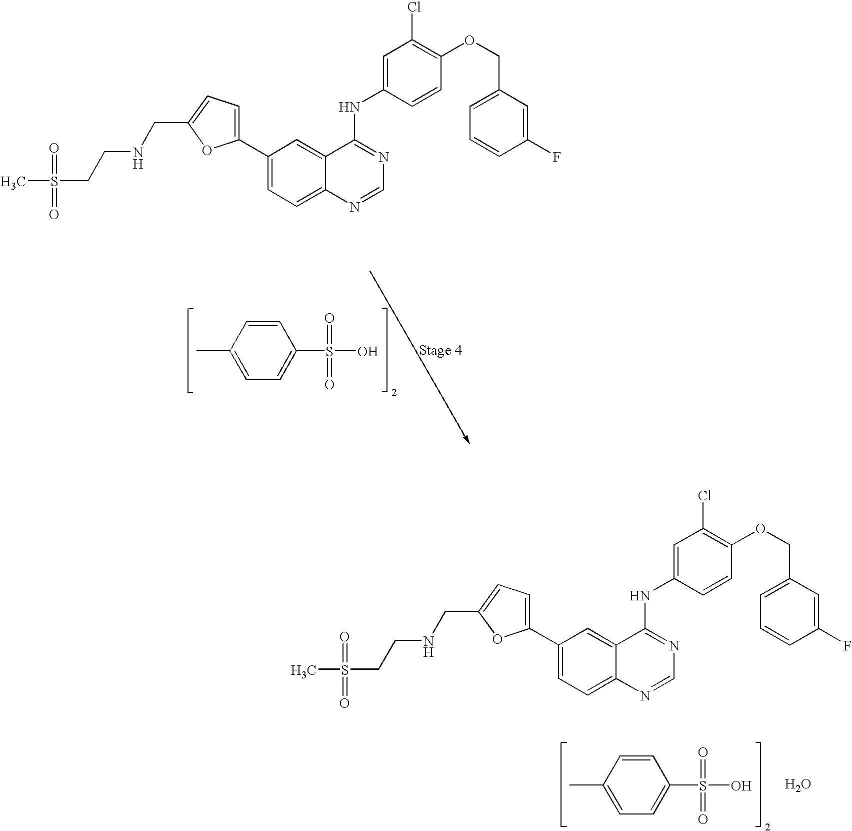

In scheme 1, the preparation of the ditosylate salt of the compound of formula (III) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

Examples of how to prepare fusion proteins of component (b) are described in WO00/44899 (US2002/0177567).

The methods, uses, combinations and compositions of the present invention may be useful for administering to a patient afflicted with cancer, eg. breast, ovarian, colon, lung or prostate cancer. In one embodiment, the cancer is an erbB2-overexpressing cancer, such as erbB2 overexpressing breast cancer. As used herein, “treating cancer” does not require that the subject be cured. As will be apparent to one skilled in the art, successful clinical outcomes when treating cancer include longer survival time, longer time to disease progression, partial response, as well as remission of the cancer.

### Use of Survivin as a BioMarker

The results provided herein indicate that enhanced tumor cell apoptosis in erbB2-overexpressing cancer cells following combined therapy (using a dual EGFR/erbB2 inhibitor such as lapatinib and vaccine-induced anti-Her-2/neu antibodies) was more closely associated with down-regulation of survivin protein, than with down-regulation of either MAPK-Erk1/2 or PI3K-Akt pathways. This provides a biological rationale for using such a combined therapy, and for using survivin levels as a biomarker for the efficacy of such combined therapies.

Survivin is a member of the inhibitor of the apoptosis family of proteins (LAP). Survivin protein expression was inhibited in BT474 cells treated with lapatinib alone (FIG. 12A, bottom); in contrast, pAb or trastuzumab had little effect on survivin (FIG. 12A, bottom). Combining lapatinib with either pAb or trastuzumab inhibited survivin to a greater degree (FIG. 12A, bottom) in BT474 cells. Similarly, apoptosis was enhanced in BT474 cells treated with combined lapatinib and pAb or trastuzumab, compared to lapatinib alone (FIG. 12A, top). The results presented herein using SKBR3 cells show a similar relationship between survivin inhibition and enhanced apopotosis following combined therapy, though to a lesser degree than in BT474 cells (FIGS. 16 & 17).

Thus the present results indicate that down-regulation of survivin in response to the combination of lapatinib and anti-ErbB2 antibodies is correlated with enhanced apoptosis (compared to down-regulation of pErk1/2 or pAkt). In the present examples, treatment conditions that inhibited survivin protein the most (lapatinib combined with either pAb or trastuzumab) resulted in marked tumor cell apoptosis.

Thus a further embodiment of the present invention relates to the use of survivin as a biomarker in the treatment of cancer with dual EGFR/erbB2 kinase inhibitors, such as the compounds of component (a) described herein.

Changes in the levels of various specific proteins in tumor tissue in response to treatment with a dual EGFR/erbB2 tyrosine kinase inhibitor have been suggested as useful in assessing whether a patient's tumor is responding to that therapy. See e.g., WO2005/017493; WO2004/000094. The present results indicate that survivin levels in erbB2-overexpressing tumor cells or tissues can be used to predict the likelihood that a patient with such a tumor will respond favorably to treatment with a dual EGFR/erbB2 tyrosine kinase inhibitor as described herein, or to treatment with a combination of component (a) and (b) as described herein. Subjects whose tumor cells have decreased levels of survivin after an initial period of treatment (decreased compared to pre-treatment levels) are more likely to have a favorable clinical response to such treatment than subjects with unchanged, or increased, levels of survivin in tumor cells following an initial period of treatment. In contrast, subjects with unchanged or increased levels of survivin after an initial period of treatment are less likely to have a favorable clinical response, and may benefit from alternate treatments.

Survivin may be measured by any suitable means as is known in the art. As used herein, a subject or patient refers to a mammal, including humans, afflicted with a solid tumor that overexpresses erbB2. As used herein, a ‘favorable clinical response’ to a treatment refers to a biological or physical response that is recognized by those skilled in the art as indicating a decreased rate of tumor growth, compared to tumor growth that would occur in the absence of any treatment; it is not meant to indicate a cure. Pre-treatment levels of survivin are assessed within a biologically relevant period prior to treatment, preferably within one month, two weeks, ten days, or one week prior to treatment. After an initial treatment period has passed, survivin levels in the tumor tissue are re-assessed.

### Examples

The invention will now be described further, with reference to the following non-limiting examples:

### Example 1

### Introduction

As shown by the following examples, vaccination of animals with the recombinant dHER2 protein induced a polyclonal antibody response specific for the ER2/neu molecule.

The experiments described herein have shown that the polyclonal antibodies inhibit the growth of HER2 over-expressing cells in in vitro models.

The examples provided herein evaluated the anti-proliferative effect of these vaccine-generated, anti-HER2 polyclonal antibodies (pAb) in combination with Lapatinib, a tyrosine kinase inhibitor specific for both the HER2/neu and EGFR molecules, on human breast cancer cells over-expressing the HER2/neu molecule (BT474 and SKBR3).

### Methods and Materials

### Example 2

### Construction of Expression Plasmid Coding for the HER2/neu Protein

A stable CHO-K1 cell line, producing a recombinant Her2/neu glycoprotein has been established. The protein is secreted into the cell culture medium, from which it is subsequently purified.

Briefly, the cDNAs corresponding to the extracellular domain of the HER2/neu molecule (ECD: AA 1-653) and to the phosphorylation region (PD) of the intracellular domain (ICD: AA 991-1255) were amplified by RT-PCR (Clontech) starting with mRNA prepared from a breast tumour sample. The 2 cDNAs were then ligated and cloned into a pcDNA3.1 hygromycin vector and a HindII-XbaI restriction fragment (2782 bp long) was cloned into an CHO K1-Glutamine Synthase expression vector (pEE14 I Celltech ) This plasmid was named pEE14-ECD-PD#13 also named pRIT 15050 and used to stably transfect CHO-K1 cells—-(derived from Lonza's MCB 024M), using the classical CaPO4 co-precipitation procedure. Transfected clones were selected in GMEM (Glasgow Minimal Eagle's medium) without Glutamine, supplemented with 5% foetal bovine serum (New Zealand) glutamate, asparagine, nucleosides and 30 μM MSX as selective reagent. One clone (#13002) was selected based on its expression level. The recombinantly expressed protein is a truncated version of the HER2/neu growth factor receptor consisting of a fusion of the extracellular domain (ECD) and a C-terminal phosphorylation part (PD) of the intracellular domain (ICD), excluding the transmembrane portion and the phosphokinase moiety of the receptor. This recombinant dHER2 protein is efficiently secreted from CHOK1 cells and is purified from the cell culture supernatant.

### Example 3

HER2/neu Protein Purification (dHER2)

The culture harvest was subjected to chromatographic separation on an anion exchange Q Sepharose FF column (Amersham) equilibrated in a 20 mM Bis-Tris propane buffer pH 6.5 containing 150 mM of sodium chloride (NaCl). Antigen was eluted from the column by increasing the concentration of NaCl in the same equilibration buffer. After addition of phosphate, antigen positive eluate was passed through 2 successive affinity columns, one Macro-Prep Ceramic Hydroxyapatite type I column (Bio-Rad) equilibrated in a 40 mM phosphate buffer pH 7.0 followed by one Blue Trisacryl plus LS column (Biosepra) equilibrated in a 10 mM phosphate buffer pH 7.0. After addition of ammonium sulphate (AMS) and pH adjustment (7.0), Blue Trisacryl flow-through containing the antigen was then injected onto an hydrophobic Ether Toyopearl 650 M column (TosoHaas) equilibrated in a 10 mM phosphate buffer pH 7.0 containing 1.2 M AMS. Antigen was eluted from the column by decreasing the concentration of AMS in the same phosphate buffer. Antigen positive eluate was concentrated then diafiltered against the final 5 mM phosphate buffer pH 7.0 on a Biomax 10 kDa membrane (Millipore). Ultrafiltration retentate was submitted to a nanofiltration step (Planova 15 N membrane, Asahi) and the resulting permeate was then sterile filtered through a 0.22 μm Durapore membrane (Millipore). Purified material was stored at −20° C.

### Example 4

### Vaccine or Immunogenic Composition

The immunogenic composition used in the present examples comprised the ECD-PD (known as a “deleted” construct of Her-2/neu, or “dHER”) protein formulated extemporaneously, or the day before injection with a GSK Bio proprietary adjuvant system comprising a liposomal formulation of 50 μg 3D-MPL (Corixa, Seattle, Wash., USA), 50 μg QS21 (Antigenics, New York, N.Y., USA) admixed with 500 μg CpG Oligonucleotide 2006 (also known as ODN 7909) (Coley Pharmaceutical).

### Example 5

### Cell Lines and Reagents

BT474 and SKBR3 cells were obtained from American Type Culture Collection (Manassas, Va., USA) and were cultured in DMEM supplemented with 10% Fetal Bovine serum.

Anti-human survivin antibody was from RD System (Minneapolis, Minn., USA). Antimouse IgG was from Rockland (Gilbertsville, Pa., USA). Antibodies to phosphotyrosine and actin were purchased from Sigma-Aldrich (St Louis, Mo., USA). Anti-ErbB1 (Ab-12) and anti-ErbB2 (Ab-11) antibodies were from Neo Markers (Union City, Calif., USA). Anti-phospho-AKT (Ser 437) was from Cell Signaling Technology, Inc. (Beverly, Mass., USA). Anti-Akt1/2, anti-phospho-Erk1/2, anti-Erk1 and anti-Erk2 antibodies were purchased from Santa Cruz Biotechnology, Inc. (Santa Cruz, Calif. USA). Trastuzumab was purchased from Genentech Inc. (South San Francisco, Calif., USA). Guava PCA 96 Nesin kit was purchased from Guava Technologies Inc. (Hayward, Calif., USA). SuperSignal West Femto Maximum Sensitivity Substrate was from Pierce (Rockford, Ill., USA). Protein G agarose was purchased from Boehringer Mannheim (Germany). GW572016 (lapatinib) was synthesized as described (Cockerill et al., Bioorg Med. Chem. Lett, 11:1401-1405 (2001)). Lapatinib for cell culture work was dissolved in DMSO (Xia et al., Oncogene 21:6255-63 (2002)).

### Example 6

### Animals

Female New Zealand White rabbits vaccinated with the dHER2 protein in adjuvant were used as source of serum for the in vitro growth inhibition experiments. Briefly the rabbits were vaccinated intramuscularly 3 weeks apart with 100 μg of dHER2 protein formulated in the liposomal adjuvant system as described in Example 4. A booster injection was given at day 161 and sera were taken 14 days later. The IgG fraction of the serum was purified on protein A sepharose and concentrated to 10 mg/ml.

The selectivity of pAb for ErbB2 over ErbB1 and ErbB3 was demonstrated by immunoprecipitation and Western blot analysis in BT474 cells (data not shown). In contrast to a monoclonal antibody such as trastuzumab, polyclonal antibodies are characterized by a spectrum of binding affinities against multiple epitopes of a target immunogen.

### Example 7

### Assessment of Apoptosis: Annexin V Staining and Flow Cytometry

Cells assessed using Annexin V staining and Flow Cytometry were treated in 6 well plates with DMSO, lapatinib, pAb, serum from pre-immunized rabbits (referred to herein as ‘pAB pre-immune’ or TA2021), Trastuzumab, and/or Gefitinib at concentrations as indicated in the figure legends of FIGS. 12A, 12C, 15A and 16. After harvesting the cells with trypsin-EDTA, 5000 cells in 50 μl were sampled on 96-well microplates. The cells were stained directly in the microplate with Annexin V-PE and Nexin 7-AAD in 1× Nexin Buffer in a 200 μl final reaction volume. After incubating for 20 minutes at room temperature, the reaction samples were ready to be acquired in the Guava PCA-96-system (Guava Technology, Inc., Hayward, Calif. USA).

7-AAD binds to cellular nuclear material after cellular membranes break down in late apoptosis. Annexin-V staining indicates early apoptotic changes that precede the loss of cellular membrane integrity. Use of Annexin-V in conjunction with 7-amino-actinomycin D (7-AAD) thus can identify both early and late apoptotic cells.

Flow cytometry results were graphed as dot plot results (not shown) with annexin V on the X-axis and 7-AAD on the Y-axis. Low annexin V and low 7-AAD represented viable cells; low annexin V and high 7-AAD indicated nuclear debris; cells with high annexin V staining indicated apoptotic cells. The graphs provided in FIGS. 12A, 12C, 15A, and 16 show the percentage of apoptotic cells (high annexin V) compared to total cells.

### Example 8

### Cellular Protein Levels: SDS-PAGE and Western Blot Analysis.

To assess the levels of various proteins in cells, whole cell extracts were prepared by scraping cells off petri dishes, washing the cell pellet twice in phosphate buffered saline (PBS), and then resuspending the pellet in two-packed-cell volumes of RIPA buffer(150 mM NaCl, 50 mM Tris-HCl, pH 7.5, 0.25% (w/v) deoxycholate, 1% NP-40, 5 mM sodium orthovanadate, 2 mM sodium fluoride, and a protease inhibitor cocktail). Protein concentrations were determined using a modification of the Bradford method (Bio-Rad Laboratory). The cell lysate was cleared by centrifugation; and ErbB1, ErbB2 and ErbB3 were separately immunoprecipitated from 200 μg of the lysate with 3 μg of anti-ErbB1 (Ab-13, NeoMarkers), anti-ErbB2 (Ab-11, NeoMarkers), anti-ErbB3 (C-17, Santa Cruz Biotechnology) antibodies, or 5 μg of pAb and 20 μl of protein A+G-Sepharose. The immunoprecipitates were separated by 4-15% gradient SDS-polyacrylamide agarose gel electrophoresis (SDS-PAGE), and ErbB receptors were detected by Western Blot with anti-ErbB receptor antibodies.

Steady state levels of Survivin, total ErbB2, phosphorylated ErbB2 (pErbB2 or pTyr), total Erk1/2 , activated Erk1/2 (p-Erk), total pErbB3, activated pErbB3 (pErbB3), total AKT protein and activated Akt (p-Akt) protein were assessed by Western blot. Levels of pErbB2 were assessed using an antibody that bound to multiple tyrosine-phosphorylated forms of ErbB2 (i.e., the antibody was not phosphorylation-site specific). Actin steady state protein levels served as a control for equal loading of protein.

For Western blot, equal amounts of proteins were resolved by either 7.5% or 4-15% gradient SDS polyacrylamide gel electrophoresis under reducing conditions. Proteins were transferred to Immobilon-P or nitrocellulose membranes. Efficiency and equal loading of proteins was evaluated by Ponceau S staining. Membranes were blocked for 1 hr in TBS (25 mM Tris-HCl, pH 7.4, 150 mM NaCl, 2.7 mM KCl) containing 4% (w/v) lowfat milk or 3% BSA (w/v). Membranes were then probed with specific antibodies recognizing target proteins which were visualized with the SuperSignal West Femto Maximum sensitivity substrate kit (Pierce) or Odyssey Infrared Imaging System (LI-COR, Lincoln, Nebr. USA).

### Example 9

### In Vitro Growth Inhibition Assay (Anti-Proliferation)

The antiproliferative effect of vaccine induced anti HER2 polyclonal antibodies (pAb) in combination with Lapatinib was evaluted on human breast cancer cells overexpressing the HER2/neu molecule (BT474 and SKBR3 cells) using the [³H] thymidine incorporation method. Briefly, 10e4 cells were seeded in 200 μl, in triplicate, in 96-w plates and incubated for 72 h at 37° C. in
1) medium;
2) medium containing Lapatinib alone (0.01 μM);
3) medium containing pAb alone (50 μg/ml);
4) medium containing pre-immune pAb (TA2021) (50 μg/ml);
5) a combination of (2) Lapatinib (0.01 μM ) and (3) pAb (50 μg/ml); or
6) a combination of (2) Lapatinib (0.01 μM ) and (4) pre-immune pAb (TA2021) (50 μg/ml).

1 μCi of [³H] Thymidine (5 Ci/mmol; Amersham) was added for an additional 24 h after which cells were harvested by trypsinisation onto filter plates and the incorporated radioactivity was counted in a beta counter. Results were expressed in cpm and the percentage of growth inhibition was calculated referring to the medium.

Dose range curves were established for both Lapatinib and pAb (data not shown). Suboptimal doses of each component, inducing only 10 to 30% growth inhibition were chosen in order to be able to see potential additive or synergistic effects.

Purified IgG from non vaccinated rabbits (pAb pre-imm) were used as negative controls in these assays.

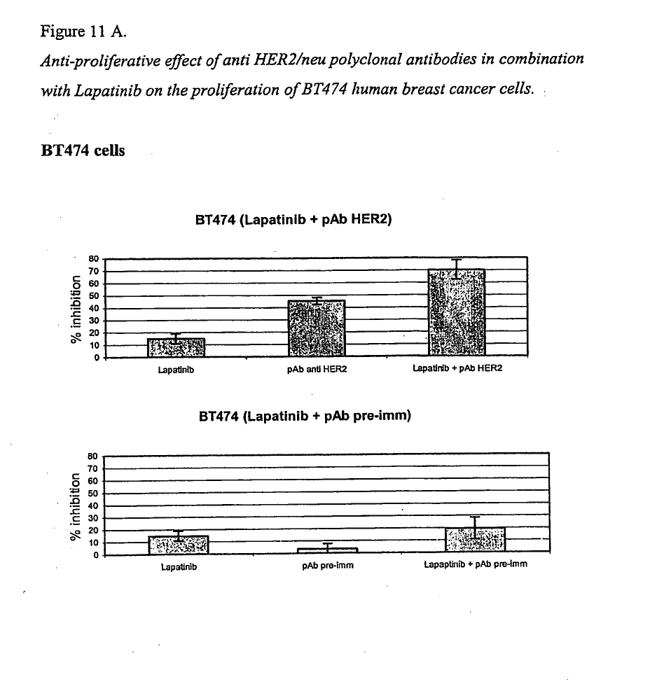

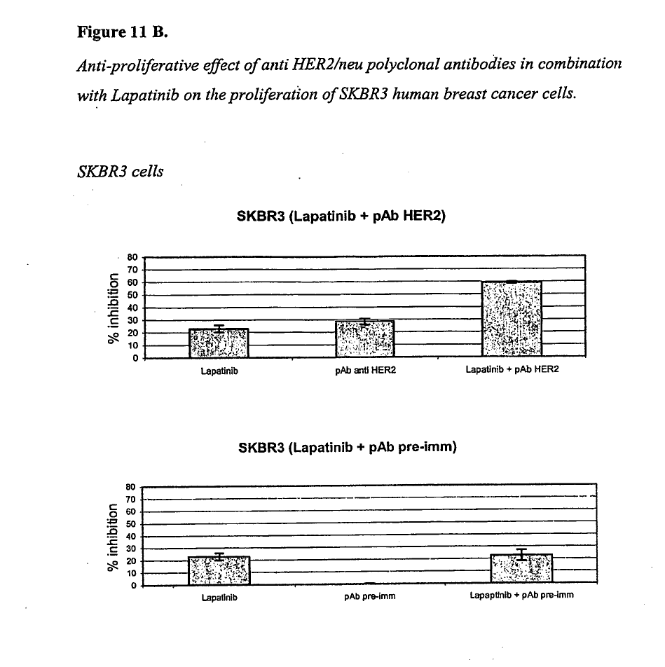

The experiments have been performed at least three times independently and the combined data for the triplicate wells of one representative experiment are provided in FIGS. 11A and 11B.

### Example 10

### Results: Antiproliferative Effect

Vaccination of rabbits with the recombinant dHER2 protein formulated in a strong adjuvant induced a polyclonal antibody response specific for the HER2/neu molecule.

The antiproliferative effect of these anti HER2 polyclonal antibodies (pAb) in combination with Lapatinib, a tyrosine kinase inhibitor specific for both the HER2/neu and EGFR molecules, was evaluated on human breast cancer cells over-expressing the HER2/neu molecule (BT474 and SKBR3). Data are shown in FIGS. 11A and 11B

### Example 11

### Results (Apoptosis and Protein Levels)

All results as shown in FIGS. 12A, 12C, 12D, 13, 14, 15A, 15B, 15C, 16 and 17 are representative of at least three independent experiments.

As can be seen in FIGS. 12A, B and C, Lapatinib and vaccine-induced anti-HER-2/neu antibodies (pAb) synergize to induce apoptosis of ErbB2 (HER-2/neu) over-expressing BT474 cells.

FIG. 12A shows the results of treating exponentially growing BT474 with (i) DMSO alone (negative control for lapatinib) (ii) lapatinib (0.1 μM), (iii) pAb (100 μg/ml), (iv) lapatinib and pAb, (v) TA2021 (100 μg/ml), (vi) lapatinib and TA2021, (vii) trastuzumab (10 μg/ml), or (viii) lapatinib and trastuzumab After 72 hr, apoptosis was assessed using annexin V staining and flow cytometry. Steady state protein levels of activated phospho-ErbB2 (p-ErbB2), total ErbB2, and survivin were also assessed after 72 hr using Western blot (FIG. 12A, bottom panel). Actin steady state protein levels served as a control for equal loading of protein. BT474 cells treated with vehicle (DMSO) or TA2021 served as controls for lapatinib and pAb, respectively.

FIG. 12B shows the effects of varied treatment conditions on BT474 cell growth (after 72 hr) using contrast phase microscopy. Treatment conditions included those as described for FIG. 12A, and additionally, gefitinib (Iressa) (0.1 μM); gefitinib (0.1 μM) and pAB (100 μg/ml); gefitinib (0.1 μM) and TA2021 (100 μg/ml); and gefitinib 0.1 μM and trastuzumab (10 μg/ml).

FIG. 12C shows the effects of increasing concentrations of pAb either alone or in combination with lapatinib (100 nM) on apoptosis in BT474 cells using annexin V staining and flow cytometry.

FIG. 12D shows steady-state protein levels of total ErbB2 and p-ErbB2 in cells treated with increasing conditions of pAb either alone or in combination with lapatinib (100 nM), assessed by Western blot. Actin steady state protein levels serve as a control for equal loading of protein.

FIG. 13 shows the activation-state of Erk1/2 and Akt is modulated in response to lapatinib and vaccine-induced anti-HER-2/neu antibodies (pAb). Exponentially growing BT474 cells were cultured for 72 hr under the treatment conditions described. Steady state protein levels of total Erk1/2, activated phospho-Erk1/2, total Akt, and activated phospho-Akt were assessed by Western blot. Cells treated with either vehicle (DMSO) or TA2021 alone served as controls.

FIG. 14 shows the effects of lapatinib and vaccine-induced anti-HER-2/neu antibodies (pAb) on the activation state of ErbB3. BT474 cells were cultured under various treatment conditions, as shown. After 72 hr, cell lysates were collected and steady state protein levels of total ErbB3 and activated phospho-ErbB3 assessed by Western blot.

FIG. 15 shows the differences between lapatinib and gefitinib in their ability to synergize with vaccine-induced anti-HER-2/neu antibodies (pAb) to induce BT474 cell apoptosis and to modulate survivin. Gefitinib (Iressa or zd1839) is an inhibitor of the EGFR protein tyrosine kinase.

FIG. 15A shows results using BT474 cells in exponential growth phase, cultured for 72 hr under the indicated treatment, conditions. Apoptosis was assessed using annexin V staining and flow cytometry.

FIG. 15B shows results of Western blot analysis of steady state protein levels of total ErbB2, p-ErbB2, and survivin after 72 hr in BT474 cells cultured under the indicated treatment conditions.

FIG. 15C shows effects of the indicated treatment conditions on steady state protein levels of total Erk1/2, p-Erk1/2, total Akt, and p-Akt in BT474 cells, assessed by Western blot after 72 hr of treatment.

FIG. 16 shows effects of the indicated treatment conditions on SKBR3 cells. After 72 hr, apoptosis was assessed using annexin V staining and flow cytometry.

FIG. 17 shows the effects of the indicated treatment conditions on survivin in SKBR3 cells after 72 hours of treatment.

FIG. 18 shows the effects of lapatinib and anti-Her-2/neu antibodies on pTyr/ErbB12 and down-stream biomarkers in SkbR3 cells, as indicated.

As shown in the Figures discussed above, treatment with lapatinib, pAb, or trastuzumab as monotherapies resulted in a slight increase in apoptosis (FIG. 12A top panel) compared to controls (DMSO, TA2021). Combining lapatinib with either pAb or trastuzumab resulted in enhanced tumor cell apoptosis compared with either treatment alone (FIG. 12A, top panel). Increasing the concentration of pAb by itself had little impact on apoptosis (FIG. 12C). When combined with a fixed concentration of lapatinib, increasing the concentration of pAb resulted in enhanced B474 cell apoptosis (FIG. 12C). Similarly, combining lapatinib with pAb also enhanced apoptosis of SKBR3 cells, another ErbB2-overexpressing breast cancer cell line, although less than that observed in BT474 cells (FIG. 16).

As also shown in the Figures discussed above, steady-state levels of activated, phosphorylated ErbB2 protein (pErbB2) were inhibited by lapatinib alone, without affecting total ErbB2 protein (FIG. 12A, bottom panel). Lapatinib also reduced steady state levels of pErk1/2 and pAkt (FIG. 13). In contrast, pAb markedly inhibited total ErbB2 protein without affecting phosphorylated (activated) ErbB2 (FIG. 12A, bottom panel). Phospho-Erk1/2 and pAkt were also markedly inhibited by pAb (FIG. 13). In the present studies, trastuzumab had less effect on total ErbB2 compared with pAb, although it slightly inhibited pErbB2 (FIG. 12A, bottom panel). Additionally, pErk1/2 and pAkt were also inhibited by trastuzumab, although less than that observed with pAb (FIG. 13). Combining lapatinib with pAb resulted in further inhibition of pErbB2 compared with lapatinib alone (FIG. 12A, bottom panel). The combination of lapatinib with trastuzumab completely inhibited pErbB2 steady-state protein levels (FIG. 12A, bottom panel), in addition to inhibiting pErk1/2 and pAkt.

Increasing the concentration of pAb from 20 to 100 to 200 μg/ml did not lead to increased inhibition of total ErbB2 protein but did result in enhanced inhibition of pErbB2, especially at higher concentrations of pAb.

### Conclusion

As shown in FIGS. 11A and B, using 50 μg/ml of vaccine-induced anti-HER2/neu polyclonal serum and 0.01 μM of Lapatinib, respectively, only modest growth inhibition on both cell lines was observed.

Combining vaccine-induced anti HER2/neu antibodies (pAb) and Lapatinib resulted in more pronounced growth inhibition than single agent treatment. This combination shows synergistic (greater than additive) growth inhibitory effects on both BT474 and SKBR3 Her2/neu over-expressing cell lines.

What is claimed is:

1. A method of treating cancer in a mammal, comprising: administering to said mammal a therapeutically effective amount of (a) a compound selected from the group of compounds of formula I, formula II, formula III, formula IV, salts of formula I, salts of formula II, salts of formula III, salts of formula IV, solvates of formula I, solvates of formula II, solvates of formula III, and solvates of formula IV, in which R₁ is Cl or Br; X is CH, N, or CF; and Het is thiazole or furan; and
(b) an immunogenic composition comprising an isolated protein comprising at least one epitope from the HER-2/neu protein, or a polynucleotide encoding such an isolated protein.

2. The method of claim 1, wherein R₁ is Cl; X is CH; and Het is furan.

3. The method of claim 1, wherein R₁ is Br; X is CH; and Het is furan.

4. The method of claim 1, wherein R₁ is Cl; X is CH; and Het is thiazole.

5. The method of claim 1, wherein component (a) is a compound of formula II.

6. The method of claim 1, where component (a) and component (b) are administered simultaneously.

7. The method of claim 1 where component (a) and component (b) are administered sequentially.

8. A pharmaceutical composition comprising therapeutically effective amounts of:
(a) a compound selected from the group of compounds of formula I, formula II, formula III, formula IV, salts of formula I, salts of formula II, salts of formula III, salts of formula IV, solvates of formula I, solvates of formula II, solvates of formula III, and solvates of formula IV, in which R₁ is Cl or Br; X is CH, N, or CF; and Het is thiazole or furan; and
(b) an immunogenic composition comprising an isolated protein comprising at least one epitope from the HER-2/neu protein, or a polynucleotide encoding such an isolated protein.

9.-12. (canceled)

13. A method according to claim 1, in which the immunogenic composition comprises a fusion protein comprising a HER-2/neu extracellular domain fused to a HER-2/neu phosphorylation domain.

14. A method according to claim 1, in which the immunogenic composition comprises a fusion protein comprising a HER-2/neu extracellular domain fused to a HER-2/neu intracellular domain.

15. A method according to claim 1, in which the immunogenic composition comprises an adjuvant.

16. A method according to claim 15, in which the adjuvant comprises one or more of cholesterol; oil-in-water emulsion; oil-in-water emulsion low dose; tocopherol; liposome; a saponin; 3D-MPL, and an immunostimulatory oligonucleotide.

17. A method according to claim 16, in which the adjuvant comprises a saponin, together with an immunostimulatory oligonucleotide.

18. A method according to claim 16, further comprising a lipopolysaccharide.

19. A method according to claim 16, wherein the saponin is QS21.

20. A method according to claim 18, wherein the lipopolysaccharide is selected from the group of
(i) Monophosphoryl Lipid A;
(ii) 3-0-Deacylated Monophosphoryl Lipid A; and
(iii) Disphosphoryl Lipid A.

21. An immunogenic composition according to claim 33 wherein the immunostimulatory oligonucleotide contains at least two CpG motifs.

22. An immunogenic composition according to claim 33 wherein the immunostimulatory oligonucleotide is selected from the group:
SEQ ID No 22
TCC ATG ACG TTC CTG ACG TT (CpG 1826),
SEQ ID No 23
TCT CCC AGC GTG CGC CAT (CpG 1758),
SEQ ID No 24
ACC GAT GAC GTC GCC GGT GAC GGC ACC ACG,
SEQ ID No 25
TCG TCG TTT TGT CGT TTT GTC GTT (CpG 2006),
and
SEQ ID No 26
TCC ATG ACG TTC CTG ATG CT (CpG 1668).

23. A composition according to claim 33 wherein the saponin is formulated to form ISCOMS or liposomes.

24. A composition according to claim 33 wherein the saponin is present in an oil in water emulsion.

25. A method according to claim 1, in which the at least one epitope from the HER-2/neu protein is from the extracellular region of the HER-2/neu protein.

26. The composition of claim 8, wherein R₁ is Cl; X is CH; and Het is furan.

27. The composition of claim 8, wherein R₁ is Br; X is CH; and Het is furan.

28. The composition of claim 8, wherein R₁ is Cl; X is CH; and Het is thiazole.

29. The composition of claim 8, wherein component (a) is a compound of formula II.

30. The composition of claim 8, in which the immunogenic composition comprises a fusion protein comprising a HER-2/neu extracellular domain fused to a HER-2/neu phosphorylation domain.

31. The composition of claim 8, in which the immunogenic composition comprises a fusion protein comprising a HER-2/neu extracellular domain fused to a HER-2/neu intracellular domain.

32. The composition of claim 8, in which the immunogenic composition comprises an adjuvant.

33. The composition of claim 32, in which the adjuvant comprises one or more of cholesterol; oil-in-water emulsion; oil-in-water emulsion low dose; tocopherol; liposome; a saponin; 3D-MPL, and an immunostimulatory oligonucleotide.

34. The composition of claim 33, in which the adjuvant comprises a saponin, together with an immunostimulatory oligonucleotide.

35. The composition of claim 33, further comprising a lipopolysaccharide.

36. The composition of claim 33, wherein the saponin is QS21.

37. The composition of claim 35, wherein the lipopolysaccharide is selected from the group of
(i) Monophosphoryl Lipid A;
(ii) 3-0-Deacylated Monophosphoryl Lipid A; and
(iii) Disphosphoryl Lipid A.
